(19) United States
(12) Patent Application Publication
(10) Pub. No.: US 2016/0358295 A1
(43) Pub. Date: Dec. 8, 2016

(54) SYSTEM AND METHOD FOR ANALYZING A MEDICAL NETWORK

(71) Applicants: James Heffley, Louisville, KY (US); Dilpreet Bawa, Louisville, KY (US); Sarah Rogers, Louisville, KY (US)

(72) Inventors: James Heffley, Louisville, KY (US); Dilpreet Bawa, Louisville, KY (US); Sarah Rogers, Louisville, KY (US)

(73) Assignee: HUMANA INC., Louisville, KY (US)

(21) Appl. No.: 13/286,935

(22) Filed: Nov. 1, 2011

(51) Int. Cl. G06Q 50/22

(52) U.S. Cl. CPC G06Q 50/24; A61B 34/10

(57) ABSTRACT

A computerized system and method for analyzing medical network adequacy. The computerized system and method uses agency guidelines and location information to generate a report as to whether a network of health care service providers satisfies adequacy requirements or access parameters and further identifies why access parameters were not met according to specialty within a county. It further identifies potential health care service providers for addition to the network to improve the compliance of the network. The analysis extends beyond pass-fail compliance with access parameters as determined by a healthcare agency and quantifies the impact of the addition of a non-participating health care services provider to the network. Non-participating health care service providers may be identified in a list and further, rank ordered within the list so that the providers likely to have the greatest impact on the adequacy measure are listed first.

### CROSS REFERENCES TO RELATED APPLICATIONS

None.

### FIELD OF THE INVENTION

The present invention relates to systems and methods for measuring the adequacy of a Medicare Network. Specifically, exemplary embodiments relate to a computerized system that processes data regarding health care service providers within a Medicare Advantage Network and facilitates analysis of network adequacy.

### BACKGROUND AND SUMMARY OF THE INVENTION

Medicare is a social insurance program financed by the United States government, providing health insurance coverage to people aged 65 and over. Currently, there are three separate Medicare coverage options which are referred to as Original Medicare, Medicare Advantage, and Medicare Supplement plans. Although similar in name, these three coverage options work differently to meet the health care needs of plan participants.

Original Medicare is a fee-for-service plan. In most cases, original Medicare is available to those 65 years of age or older, those with disabilities, and people with End-Stage Renal Disease. The service is divided into categories: Medicare Part A; and Medicare Part B. Medicare Part A covers inpatient care in hospitals including critical access hospitals and skilled nursing facilities (not custodial or long term care). It also helps cover hospice care and some home health care. Certain conditions must be met to obtain these benefits. Medicare Part B covers doctors' services and outpatient care as well as some other medical services that Part A does not cover such as the services of some physical and occupational therapists and some home health care.

Original Medicare provides very basic coverage for medical expenses, so members are still responsible for costs such as deductibles and coinsurance. Medicare Advantage and Medicare Supplement plans provide additional coverage for medical care. These plans give Medicare beneficiaries the option of receiving their Medicare benefits through private health insurance plans instead of through the original Medicare plan. For people that choose to enroll in a Medicare private health plan, Medicare pays the private health plan a specified amount every month for each member. Private plans are required to offer a benefit package comparable to Medicare's and to cover everything Medicare covers, but they do not have to cover every benefit in the same way. Medicare Supplement Plans are standardized with ten levels of coverage from which to choose. These plans are often referred to as “MediGap Insurance.” In contrast, Medicare Advantage plans offer more coverage options as they are not standardized and vary greatly from plan to plan. Medicare Advantage plans are often times referred to as “Part C” plans.

The Centers for Medicare and Medicaid Services (CMS) is the branch of the U.S. Department of Health and Human Services that administers the Medicare program. CMS has issued rules of Network Adequacy for Medicare Advantage plans which establish: 1) the minimum number of health care service providers required by a plan; and 2) the maximum time and distance that may exist on average between the plan's health care service providers and the beneficiaries of the plan. The minimum/maximum criteria currently vary by specialty type (e.g., cardiology, ophthalmology, etc.) and the geography of the region.

The inclusion of health care service providers within a Medicare Advantage plan is not restricted by their physical location within a given county as CMS also permits providers to be included in a network if they serve beneficiaries within the county in question and meet Medicare's maximum time and distance requirements. These providers are referred to as “supplemental providers” for the plan in question. Similarly, Medicare allows consideration of local established patterns of care and other factors that govern reasonable access which permits additional providers to be included in a network in some instances. These providers are referred to as “pattern of care” providers.

Determining the minimum number of health care service providers required by a Medicare Advantage network requires a calculation of the average number of beneficiaries enrolled in the plan and a calculation of the enrollee to health care service provider ratio. The average number of beneficiaries enrolled in the plan is determined by multiplying the number of Medicare beneficiaries that reside in the plan County by the County's specified penetration rate:

$$Average Enrollment of Beneficiaries Served by Health Plan = ( Number of Beneficiaries Residing in County \times 95 th Percentile for County ) . \qquad ( Equation 1 )$$

The County's specified Penetration rate is dependent on how the county has been designated (i.e., large metro, metro, micro, or rural):

TABLE 1
Penetration Rates

| County Designation | 95^{th} Percentile |
|---|---|
| Large Metro | 7.0% |
| Metro | 11.6% |
| Micro | 7.4% |
| Rural | 7.2% |

It follows that a County, which has been designated as a Micro County, and that has 6,669 Medicare beneficiaries, would have an estimated (6,669×0.074)=494 beneficiaries per plan.

The minimum number of health care service providers required by a Medicare Advantage Network may then be determined by the following equation:

$$( Equation 2 )$$

$$Minimum number of health care service prociders required = ( ( \frac{Average Enrollment of Beneficiaries Served by Health Plan}{100} ) \times Minimum Provider Ratio )$$

where the minimum provider ratio is established by CMS and is again dependent on how the County has been classified (e.g., Large Metro, Metro, Micro, Rural) and also dependent on the type of health care service at issue (i.e., ophthalmology, cardiology, optometry, etc.). Working from the previous example, a Micro County having 494 beneficiaries per Advantage plan would require

$$( ( \frac{494}{1000} ) \times 0.23 Cardiologists ) = 0.113 Cardiologist .$$

Because this result is rounded up to the nearest whole number, the Medicare Advantage Network in question must have one cardiologist.

Similarly, a Medicare Advantage Network is not adequate unless a certain percentage of the plan's beneficiaries meet time and distance requirements that have been established by CMS. The current requirement is that 90% of the Network satisfy time and distance requirements. CMS has correspondingly established maximum travel time and distance criteria for most health care provider facilities and services. Below is an example of Distance and Time Criteria that has been established by CMS:

TABLE 2
Distance and Time Criteria

| Provider Type | Distance Criteria | Time Criteria |
|---|---|---|
| PCP | 10 miles | 20 minutes |
| Cardiologist | 30 miles | 30 minutes |
| SNF | 60 miles | 60 minutes |

When private companies create Medicare Advantage networks, they rely on sophisticated mapping tools that allow a computer user to see where every provider is located as well as view an indication of the member population within a targeted area. The mapping tools obtain data from the company's provider and member databases in order to get location information that is used to evaluate compliance with the CMS requirements. Though this technology makes assembling adequate Medicare Advantage Networks easier, there is a need in the art for a system which utilizes the location information to generate a report as to whether a Medicare Advantage Network satisfies the CMS adequacy requirements and that further may identify potential health care service providers for addition to the network when the generated report indicates the Network is inadequate. Further, there is a need in the art for a system which extends the analysis beyond pass-fail compliance with CMS regulations in order to assemble the network that best meets the requirements of CMS.

For example, in assembling a Medicare Advantage network, there are numerous combinations of health care service providers that may combine to create a Medicare network that satisfies CMS adequacy regulations. However, some of those combinations may result in a higher quality, higher performing network by, for example, generating more revenue than others, seeing more patients than others, providing a higher quality experience for plan members than others, etc. One exemplary embodiment utilizes health care service provider data for a specified geographical location, analyzes access parameters to determine adequacy, and when access parameters are not met, facilitates the identification of non-participating providers that could be added to the network to help meet access parameters.

In a preferred exemplary embodiment, the system comprises at least one database storing routinely updated health care service provider data and one or more servers where the one or more servers periodically retrieves the updated health care service provider data from the database, and generates at least one report comprising Medicare network adequacy measurements. In some exemplary embodiments, the system generates a list of available, non-participating health care service providers that may be selected for addition into a Medicare Advantage network, permits a user to select one or more of the available health care service providers, and generates an updated adequacy report which recalculates at least one measure of network adequacy as if the selected health care service provider were contracted to be a part of the Network. In preferred exemplary embodiments, the system-generated adequacy report(s) details measures in addition to those relating to CMS adequacy by, for example, quantifying the impact of the addition of a non-participating provider to the medical network.

### BRIEF DESCRIPTION OF THE DRAWINGS

A better understanding of the disclosed embodiments will be obtained from a reading of the following detailed description and the accompanying drawings wherein identical reference characters refer to identical parts and in which:

FIG. 1 shows an exemplary embodiment of a computerized system that may be utilized to prepare a network adequacy report;

FIG. 2 shows an exemplary embodiment of a computerized system for generating a network adequacy report that does not utilize an administrator console;

FIG. 3 shows a data flow chart for an exemplary embodiment of a computerized system for generating a network adequacy report;

FIG. 4 shows a data flow chart for an exemplary embodiment of a computerized system for generating a network adequacy report that does not comprise an administrator console;

FIG. 5 shows a context diagram for an exemplary embodiment of a computerized system for generating a network adequacy report;

FIG. 6 shows an exemplary embodiment of a county summary page that may form part of a network adequacy report generated by an exemplary embodiment of the computerized system;

FIG. 7 shows an exemplary Application Detail page that may form part of an exemplary network adequacy report;

FIG. 8 shows an exemplary summary page;

FIG. 9 shows an exemplary Summary Contract Gaps Page;

FIG. 10 shows an exemplary VP Gap Bands Page;

FIG. 11 shows an exemplary VP Counties by Specialty Page;

FIG. 12 shows an exemplary VP Spec by Counties Page;

FIG. 13 shows an exemplary County Spec Detail Page;

FIG. 14 shows an exemplary embodiment of windows which may be sent to a remote computer for display when a system user instructs the system to generate a version of the report that can be saved on a remote computer, etc.;

FIG. 15 shows an exemplary Medicare Advantage Report with an option to generate and display of a list of contracted providers;

FIG. 16 shows an exemplary dynamic Medicare Network Adequacy Report;

FIG. 17 shows an exemplary generated list of contracted health care service providers;

FIG. 18 is an exemplary generated list of health care service providers;

FIG. 19 is an exemplary generated network adequacy report with importance indicators.

### DETAILED DESCRIPTION

A Medicare Advantage Network comprises contracted health care service providers. For example, cardiologists, primary care physicians, optometrists, etc. may be contracted to form a Medicare Advantage Network. Medicare Advantage Networks are established on a county-by-county basis. Health care service providers that are physically located within a county are considered “in-county” service providers. It is not necessary, however, that the health care service providers be physically located within a county to be contracted if they otherwise meet CMS distance requirements for the county. Contracted out-of-county health care service providers are referred to as supplemental health care service providers. Health care service providers that have not been contracted to be part of a particular Medicare Advantage Network, but that could be contracted (because for example they meet CMS requirements for the county in question by being within a maximum distance requirement of a certain percentage of plan beneficiaries, because they are located within the county, etc.) are referred to as available or non-participating health care service providers.

Exemplary embodiments comprise a method of utilizing a computerized system to generate an adequacy report for at least one Medicare Advantage Network. In preferred examples, the system-generated report details whether at least one Medicare Advantage Network satisfies CMS requirements based on the network's in-county and supplemental health care service providers and may incorporate additional information which could be useful to a system user attempting to assemble an improved or optimal Medicare Advantage Network that meets access parameters. For example, the report may comprise a list of available, non-participating health care service providers for addition to a Medicare Advantage Network when the current Network does not satisfy CMS requirements. Preferably, the addition of the recommended available health care service providers to the Network (i.e., the contracting of the available health care service providers to be part of the Network) would result in the Network being adequate as measured by CMS standards.

In some embodiments, the report may rank available health care service providers for addition to a Medicare Advantage Network based on their impact on access parameters. For example, CMS may require that 90% of sample Medicare beneficiaries have access to at least one participating podiatrist within 30 miles of a specified county. A Medicare Network Adequacy report may indicate that only 45% of sample beneficiaries meet this access parameter, but there are 15 non-participating podiatrists that could help meet the access parameter. The computerized system and method determines which podiatrist/podiatry locations may have the greatest impact on meeting network access parameters. For example, adding Dr. A may increase access from 45% to 80%, whereas adding Dr. B only increases access from 45% to 48%. In such a scenario, Dr. A is ranked above Dr. B because his impact on the network access parameters is greater.

In other examples, the report may recommend the removal of a contracted health care service provider from a network because, for example, the network would be more profitable and would still pass CMS standards without the health care service provider at issue. When available health care service providers have been recommended in a network adequacy report for addition to a Medicare Advantage Network, the system may be able to generate a targeted contracting list which contains contact information for the identified available health care service providers.

In some exemplary embodiments, the system generates a list of at least one available health care service provider in conjunction with or as part of a Medicare Advantage Network Adequacy Report which is sent to a remote computer for viewing by a system user. The system may permit the user to select at least one available health care service provider from the list, and send an instruction to at least one system server to generate an updated network adequacy report which details the adequacy of the network as if the available health care service provider were part of the Network. The new report allows the system user to evaluate the impact of the addition to the access parameters.

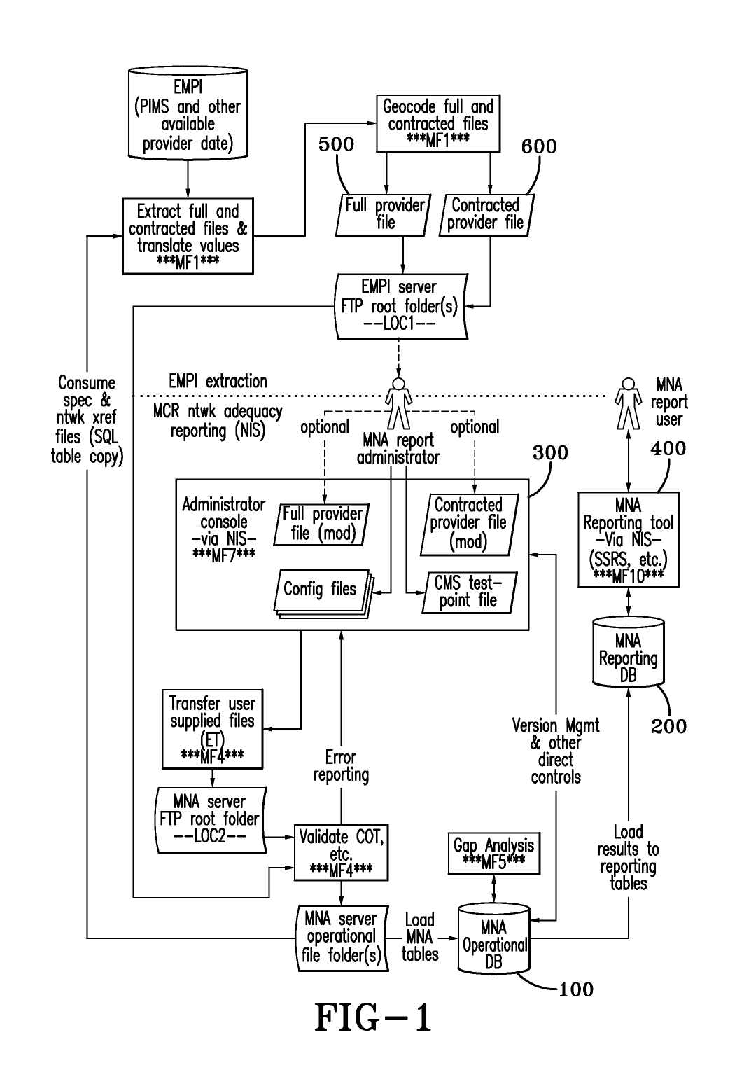

In some exemplary embodiments, such as that depicted in FIG. 1, the system generates at least one network adequacy reporting table which is utilized by the system to generate a network adequacy report displayed by a remote computer for viewing. As can be seen in FIG. 1, the computerized system may comprise an operational database 100 and a reporting database 200, where the operational database 100 stores health care service provider data and at least one measure of network adequacy which has been calculated by the system using the health care service provider data and the reporting database 200 stores at least one network adequacy reporting table which has been generated by the system and which contains at least one measure of network adequacy stored in the operational database 100, and one or more servers running an administrator console 300 which permits a system user to review, amend, and manage data and/or tables stored within the operational database 100 and also running a reporting tool 400 which is accessible by system users via the internet and which permits system users to instruct the system to generate a network adequacy report using at least one network adequacy reporting table stored in the reporting database 200.

In one exemplary embodiment, the system comprises one or more servers configured to:
Periodically receive health care service provider data for contracted and available, non-participating health care service providers;
Geocode the health care service provider data (i.e., use the service provider's address to determine the latitude and longitude of the address for each health care service provider);
Send the geocoded health care service provider data to at least one file folder depending on whether the health care service provider is contracted or available;
Validate the health care service provider data;
Load the validated health care service provider data into at least one staging table;
Send at least one staging table to an operational database for storage;
Retrieve data from a staging table stored in the operational database and utilize the data within the table to perform a gap Analysis which produces at least one measure of network adequacy (a gap analysis shows whether a Medicare Advantage Network meets CMS requirements);
Send at least one measure of network adequacy to the operational database for storage (preferably in the form of a processing table);
Retrieve at least one measure of network adequacy from the operational database and load the data and results into at least one network adequacy reporting table;
Send the network adequacy reporting table to a reporting database for storage;
Run a user interface for display by a remote computer;
Receive a request from a remote computer to view a network adequacy report; and
Retrieve a network adequacy reporting table from the reporting database and populate a formatted network adequacy report with data from the network adequacy reporting table and send the report to the remote computer for viewing.

In some exemplary embodiments of the system, the system may generate a network adequacy report upon receipt of an instruction from a remote computer to view the report. Such an exemplary system may comprise at least one database storing geocoded health care service provider data and one or more servers configured to:
Run a user interface for display by a remote computer;
Receive a request from the remote computer via the user interface to view a network adequacy report for a Medicare Advantage Network;
Retrieve health care service provider data from the database in response to the received request;
Calculate a measure of network adequacy using the retrieved health care service provider data;
Generate a network adequacy report comprising at least one measure of network adequacy; and
Send the Network Adequacy Report to the user interface for viewing.

In some exemplary embodiments, one or more servers may further be configured to:
Receive a request from the remote computer to recalculate at least one measure of network adequacy displayed within the report based on an alteration to the constitution of the network's contracted health care service providers;
Recalculate at least one measure in response to the received request; and
Send the recalculated measure(s) to the user interface for viewing at the remote computer by repopulating the network adequacy report, generating a new network adequacy report, etc.

As shown in FIG. 1, an exemplary computerized system may extract health care service provider data from the database of an entity which routinely supplies the data. Health care service provider data which may be extracted by the system may include the name and address of and type of service(s) offered by a health care service provider. Once extracted, the system preferably sends the health care service provider data to a database for storage. The data may be geocoded by the system according to geoaccess standards established by CMS and entered into a system file(s). The health care service provider data extracted by the system may include information for health care service providers that have been contracted by a private company offering at least one Medicare Advantage plan as well as information for available health care service providers.

In the exemplary embodiment shown in FIG. 1, the system comprises a full provider file 500 where the geocoded data for every health care service provider is entered (In-County, Supplemental, and Available providers) as well as a contracted provider file 600 where the geocoded data for In-County and Supplemental health care service providers may be entered. These files are preferably maintained in at least one system server. In some exemplary embodiments, once the health care service provider data has been geocoded and entered into the appropriate system file(s), the system may validate the data and send it to at least one operational database 100 for storage. In preferred exemplary embodiments, the validated data is loaded into at least one staging table and then sent to the operational database 100 for storage. Once data is stored in the operational database 100, it may be retrieved by at least one system server for the purpose of performing various calculations with the health care service provider data. For example, health care service provider data may be retrieved from the database, where the data is that of the health care service providers which have been contracted to be part of a county's Medicare Advantage Network and those that are available for the network, and at least one system server may utilize the data to determine the number of contracted health care service providers for that network as well as the number of contracted health care service providers that are located within a maximum distance requirement (typically established by CMS) of 90% of the Network's plan members. The calculations may be sent back to the operational database 100 for storage in the form of a processing table.

Because Medicare Advantage Network requirements vary based on the type of health care service at issue as well as the county where the network is located, one or more system servers may retrieve health care service provider data from a system database and make a calculation of network adequacy based on the county in which the Network is located as well as the type of health care service or specialty offered by the health care service provider(s) (primary care, allergy & immunology, cardiac surgery, cardiology, podiatry, etc.). In an exemplary embodiment, the type of health care services included in a Medicare Advantage Network depends on the type of product at issue. For example, a PPO may require a specific set of health care services. When the type of health care services included in a network depends on the type of product at issue, the system preferably determines at least one measure of network adequacy for each type of health care service required by the network.

In preferred exemplary embodiments, the system generates at least one network adequacy reporting table daily where the reporting table contains at least one measure of adequacy for more than one Medicare Advantage Network. The reporting table may be generated from at least one processing table in some exemplary embodiments, but in other exemplary embodiments the reporting table is generated by the system without first accessing a processing table. For example, the system could retrieve health care service provider data from a database and run a gap analysis on the data producing at least one measure of network adequacy and load at least one measure of network adequacy into a network adequacy reporting table which is sent to a database for storage. System users may be able to access the system, for example, through a user interface displayed by a remote computer, and send instructions to at least one system server which, in response to the instruction, retrieves at least one network adequacy reporting table from the database and populates a formatted network adequacy report with the data from the reporting table and sends the formatted report to the remote computer for viewing.

Exemplary embodiments of the system, such as that shown in FIG. 1, may comprise an administrative console 300. An administrative console may permit a system user to make amendments to health care service provider data obtained by the system and/or that which has already been geocoded and saved in system file(s), make amendments to health service delivery mapping, make amendments to CMS requirement data utilized by the system (such as for example varying the percentage of beneficiaries within a geography that must meet network access parameters, entering and modifying the distance and drive time access parameters for each health service specialty, etc.), define and edit the geographies assigned to a contracting leader, etc. In a preferred exemplary embodiment, at least one server runs an administrative console which may be accessed by a remote computer.

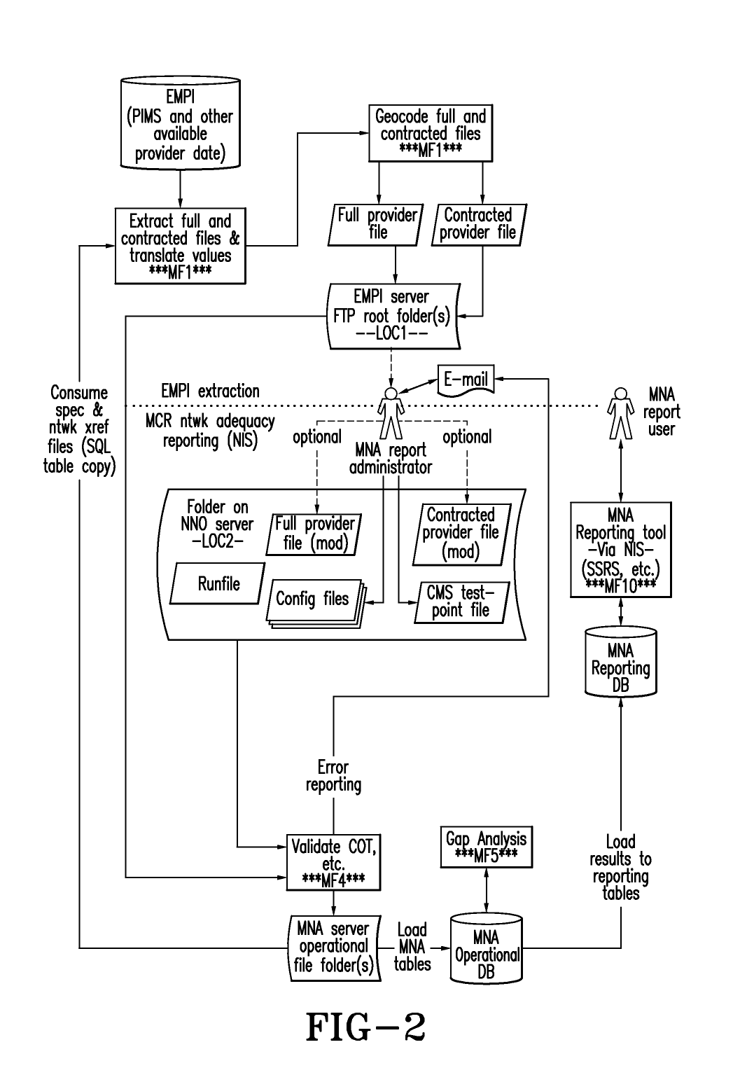

In an exemplary embodiment comprising an administrative console, at least one system server may run an administrative console interface that may be accessed by a remote computer via the internet. In such an exemplary embodiment, a system user may access the internet and reach the administrative console interface by entering the appropriate website address into the browser. Once at the website for the administrative console, the system user may be prompted for log-in credentials. The system user may enter login credentials and send them to a system server via the administrative console interface. Once the log-in credentials are received by the system server, the server may determine whether the log-in credentials are valid and if so, permit access to content of the administrative console. In one example where administrative log-in credentials have been validated, the administrative console interface permits a system user to select from a variety of administrative tasks that may be performed with the system. For example, the interface may permit a system user to send a request to view health care service provider data to a system server which in response to the request, retrieves health care service provider data from at least one system database and sends the data to a remote computer for viewing. In preferred exemplary embodiments, the health care service provider data may be viewed at the administrative console interface. Once the health care service provider data has been sent to a remote computer for viewing, a system user may be able to make amendments and/or additions to the data via the administrative console. For example, the system user may be able to utilize the administrative console interface to send a request to a system server to amend health care service provider data stored in at least one system database. In response to the request, the system server may update at least one system database with amended health care service provider data. Similarly, an administrative console may be accessed via an administrative console interface to instruct a system server to make amendments to CMS requirement data, amend health service delivery mapping data, etc. In some exemplary embodiments, a server running an administrative console application may be accessed through a network as well as or in lieu of being accessible though the internet. As can be seen in FIG. 2, some exemplary embodiments of the system do not comprise an administrator console.

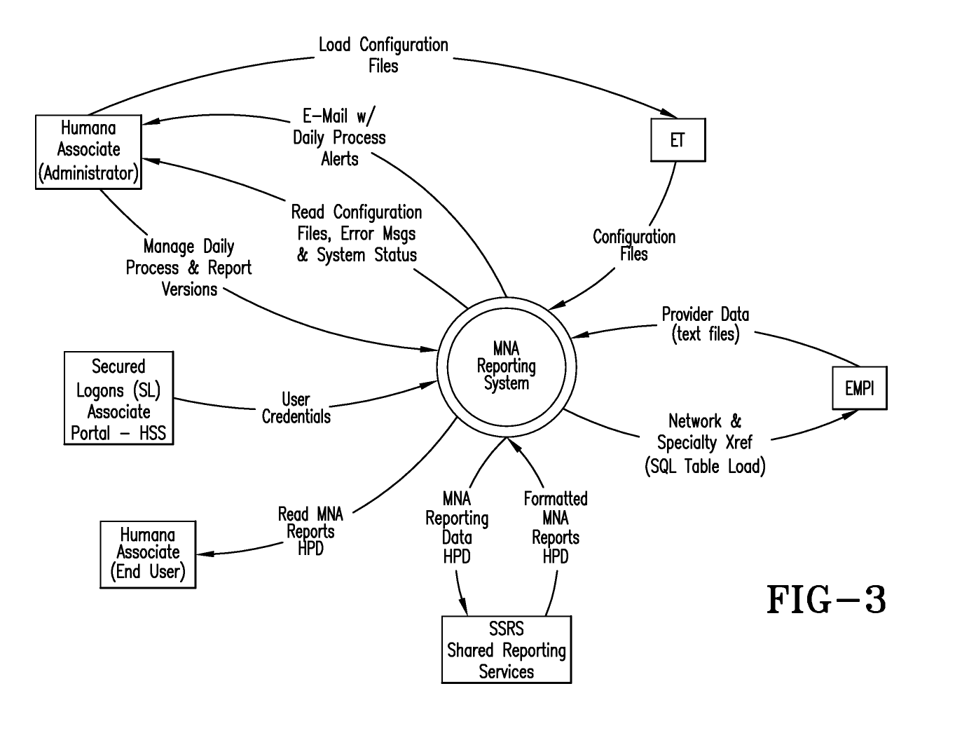

FIG. 3 shows a flow chart for an exemplary embodiment of a system and method for generating a Medicare Advantage Network Adequacy Report. As can be seen, the system may extract health care service provider data from at least one entity that supplies such data on a daily basis. Upon extracting the health care service data, the system may generate an email and cause it to be sent to a system user's email account where the email provides details as to the data extraction which has or has not taken place. For example, the email may state that the scheduled data extraction did not occur. A system user may be able to manage and load configuration files into the system by accessing at least one system server in some embodiments. Once health care service provider data has been obtained by the system, it might be processed in more than one way. In the exemplary embodiment shown in FIG. 3, the data is processed and assembled into staging tables, is subjected to a gap analysis process (determines whether a Medicare Advantage Network satisfies CMS standards), and is processed to generate a report with the results of the gap analysis into at least one network adequacy reporting table.

In exemplary embodiments which comprise an administrator console, a system user may access the system to manage the data processing of the system as well as Network Adequacy Reports generated by the system via the console. The exemplary embodiment of FIG. 3 shows how data might flow through a system comprising an administrator console. As shown, the console may be run by at least one system server and accessed by a system user via the internet. Through the console, a system user may view and process error messages that have been generated by the system during the system's configuration of Network Adequacy reporting tables and/or during the generation of Network Adequacy Reports and take steps to correct the errors identified in the messages. The system may additionally send network adequacy reporting tables and/or network adequacy reports to the administrator console for viewing and managing.

In some exemplary embodiments such as that depicted in FIG. 3 the system may utilize health care service provider data which has been extracted (obtained, downloaded, etc.) from a data provider to generate network adequacy reporting tables. These network adequacy reporting tables may then be utilized to generate at least one Network Adequacy Report which may be viewed by system users through an online interface via a remote computer. The system may generate the network adequacy reporting tables periodically in response to the extraction of health care service provider data from an external data provider. The network adequacy reports generated by the system may comprise at least one measure of network adequacy as determined by CMS regulations for at least one Medicare Advantage Network. When evaluating access parameters, the system indicates why the access parameters may not have been met, on a specialty level within a county.

In preferred exemplary embodiments, the report also comprises information which could help a system user assemble a better network. The impact of adding non-participating health care service providers is quantified to assist a user in the analysis. A system user may be able to view the network adequacy report(s) through an online interface executed by at least one server of the system once the user has entered a user ID and password and has sent instructions to the system via the interface as to which Medicare Advantage Network Adequacy Report it would like to view.

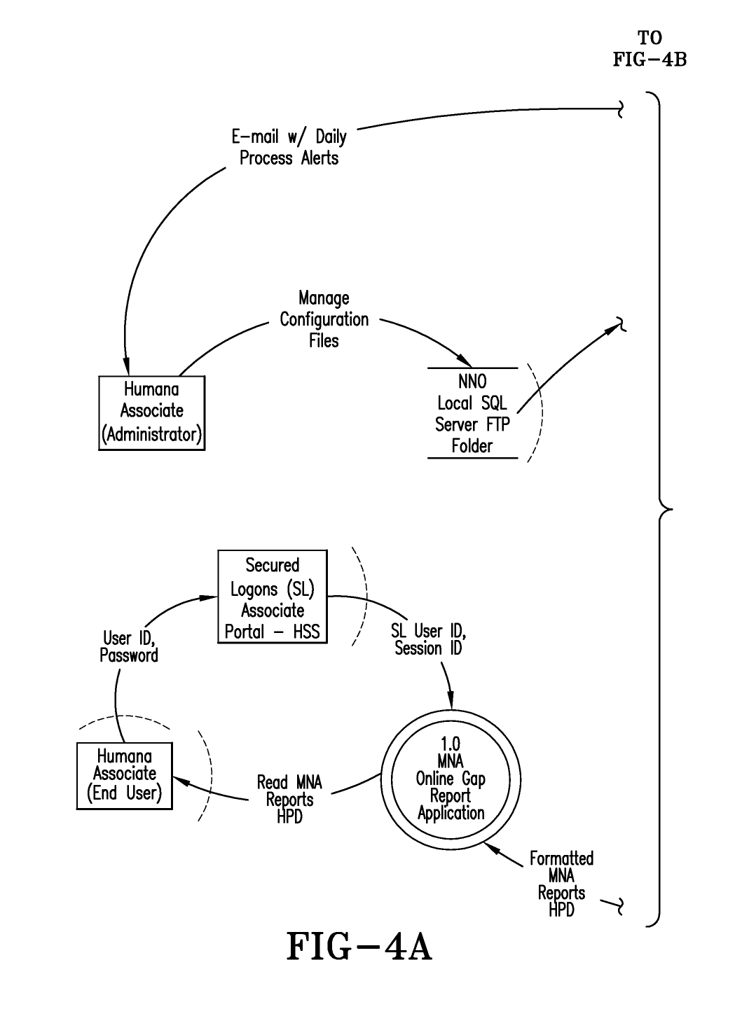

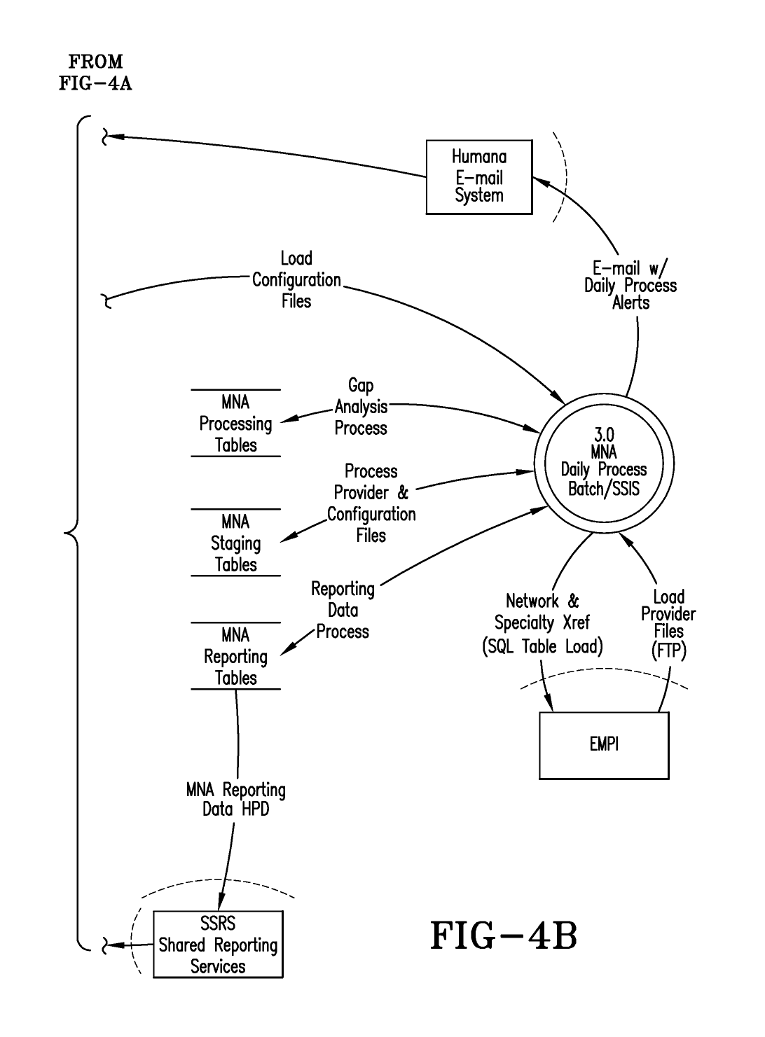

FIG. 4 also shows a flow chart for an exemplary embodiment of a system and method for generating a Medicare Advantage Network Adequacy Report. As shown in FIG. 4, data may not flow through an administrator console in some exemplary embodiments. In such embodiments, a system user may still be able to access a system server to manage configuration files and upload them into the system and may still receive e-mail process alerts that have been generated by the system. When the system does not comprise an administrator console, Network adequacy reporting tables may still be prepared by the system utilizing obtained health care service provider data. Once the report tables have been generated, at least one server may send them to a database where they can be accessed and utilized in the generation of network adequacy reports which are viewed by system users via a user interface.

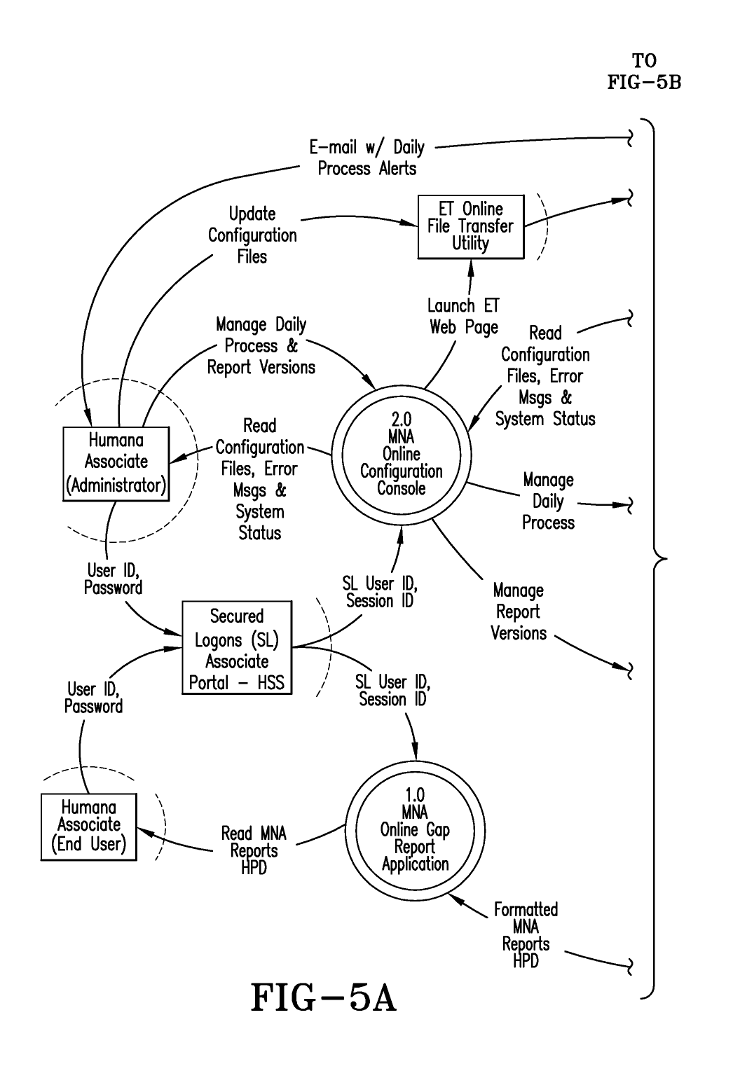

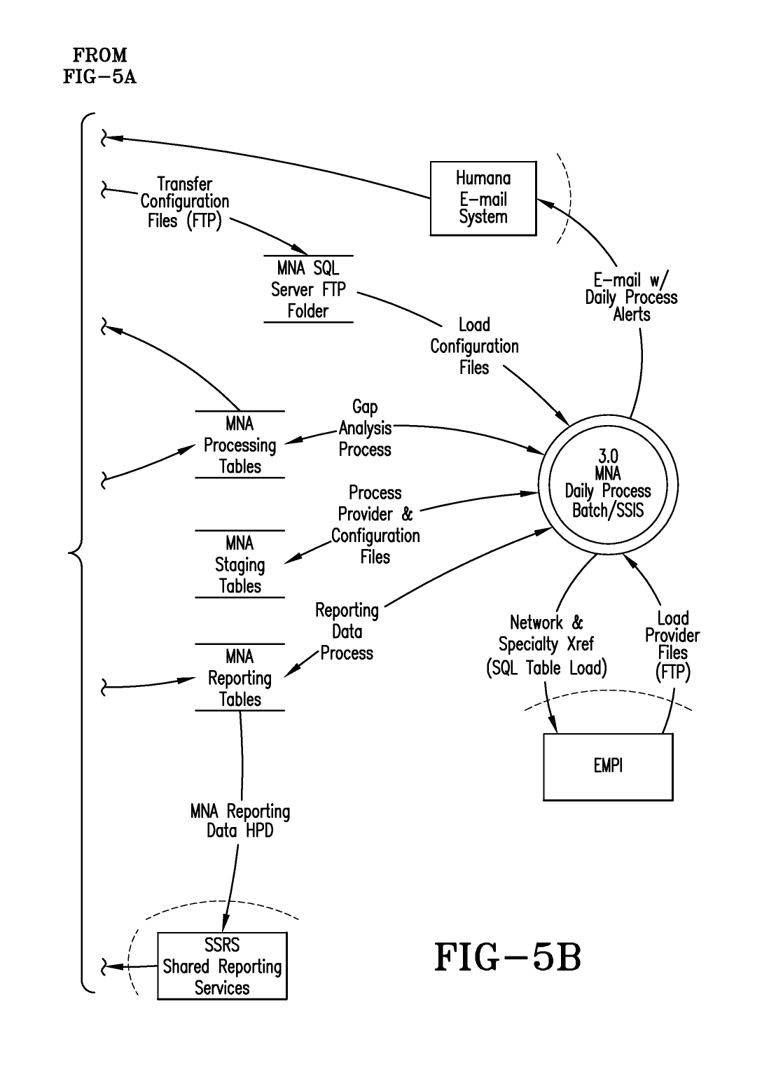

FIG. 5 also shows a flow chart for an exemplary embodiment of a system and method for generating a Medicare Advantage Network Adequacy Report. As shown in FIG. 5, a system administrator may be able to utilize the system to load configuration files, review e-mail process alerts regarding the successful or unsuccessful completion of system tasks (such as health care service provider data download, network adequacy report generation, etc.), read configuration files and error messages, check on the system's status (i.e., has the system prepared the scheduled network adequacy report(s)), and manage daily process and report versions. FIG. 5 also shows an exemplary embodiment of the system's reporting process. In the exemplary embodiment shown in FIG. 5, the system processes provider and configuration files that contain received health care service provider data and assembles staging tables and sends them to a database for storage. The system accesses the staging tables and performs a gap analysis (producing at least one measure of network adequacy) with the data. As shown in FIG. 5, the results of the gap analysis may be loaded into a processing table and sent to a database for storage. One or more system servers may access the processing table(s) and load at least one measure of network adequacy into a network adequacy reporting table which is sent to a database for storage. The staging and processing tables and network adequacy reports may be stored in a single database in some exemplary embodiments. In other exemplary embodiments, more than one database may be used. As shown, in FIG. 5, a system user may then be able to enter user credentials and cause the system to access the network reporting tables in order to generate a network adequacy report that is transmitted to a remote computer for viewing.

In one exemplary embodiment of a system and method for generating a Network Adequacy report, health care service provider data is obtained by the system on a daily basis and stored in a database that can be accessed by at least one system server. In a preferred exemplary embodiment, more than one system server can access the obtained health care service provider data. Once the system has obtained and stored the health care service provider data, it may be accessed by at least one system server and processed into at least one network adequacy reporting table. A network adequacy reporting table may comprise the data for at least one health care service provider included in a Medicare Advantage Network as well as a measure of network adequacy which has been calculated by the system. For example, the table may provide the average direct distance in miles from at least one health care service provider to network plan members, the estimated driving distance in miles from at least one health care service provider to network plan members, the estimated driving time in minutes from at least one health care service provider to network plan members, etc. In some exemplary embodiments, the system may utilize a test point to make these calculations where the test point is intended to simulate the geographical location of Medicare Advantage Network beneficiaries. The system may comprise a static file which defines Medicare Advantage Network access parameters for pass/fail by product, county, and specialty which is utilized by the system in generating at least one network adequacy reporting table. In generating at least one network adequacy reporting table in some exemplary embodiments, the system may utilize Equation 1 and/or Equation 2 to calculate at least one measure of network adequacy as measured against CMS standards. Once the system has generated at least one network adequacy reporting table, it may be electronically transmitted to a database where it may be accessed and utilized to generate a network adequacy report. In some exemplary embodiments a network adequacy report contains all the data of a network adequacy reporting table. The report is distinguished from the table in that it has been formatted for display by a remote computer. In some embodiments, a network adequacy report is generated that contains data obtained from more than one network adequacy reporting table.

Some exemplary embodiments of a system and method for generating a Network Adequacy Report comprise at least one server where the server is adapted to receive a request to view a Network Adequacy Report from a remote computer where the request comprises at least one Medicare Advantage Network Parameter, retrieve health care service provider data from a database in accordance with at least one Medicare Advantage Network Parameter, utilize the retrieved data to generate a Network Adequacy Report, and transmit the Network Adequacy Report to the remote computer for viewing. Medicare Advantage Network Parameters that may be supplied to the system include but are not limited to product, state, county, and/or specialty criteria.

In one exemplary embodiment, a system user enters a Network Parameter(s) at a remote computer and sends it to at least one server of the system. Upon receipt of the parameter(s), at least one server retrieves health care service provider data from a database, where the data corresponds to the received network parameter(s). For example, a system user may send a request to at least one system server to view a Medicare Advantage Network adequacy report for County X. Upon receipt of the request, the server may retrieve data for health care service providers that are contracted for County X's Medicare Advantage Network from at least one system database. Once the server has retrieved the relevant health care service provider data, it may utilize the data to calculate at least one measure of County X's Medicare Network adequacy, and generate a Network adequacy report comprising at least one adequacy measure which is sent to the remote computer for viewing. If the Network is inadequate according to at least one measure, the system may identify one or more available, non-participating providers that could be added to the network and provide to the user details regarding the impact of the addition of the provider to the network.

In another exemplary embodiment, the system generates at least one network adequacy reporting table(s) comprising at least one network adequacy measure and stores the reporting table(s) in a database without having received a prompt from a system user. The reporting tables are thus partially pre-processed and ready to be utilized in the generation of at least one network adequacy report upon receipt of a request from a remote computer. When the system is able to generate more than one network adequacy report, a specific Network Adequacy Report may be requested by a system user by sending identifying information, such as at least one Medicare Advantage Network Parameter, to at least one system server along with an instruction to view a corresponding Medicare Network Adequacy Report.

In some exemplary embodiments, a server receives a request to generate a Medicare Network Adequacy Report. A request to generate a Medicare Network Adequacy Report may consist of receiving at least one Medicare Advantage Network Parameter from a remote computer. The server may then retrieve health care service provider data from at least one database that corresponds to at least one Network Parameter, process the data to determine at least one measure of network Adequacy, and send the measure to the remote computer for viewing in the form of a Network Adequacy Report. A measure of network Adequacy displayed within a Network Adequacy Report may be the average direct distance from Network plan members to a health care service provider, the average driving distance from Network plan members to a health care service provider, the average time in minutes from Network plan members to a health care service provider, the percentage of Network Plan members that meet accessibility requirements as established by CMS, etc.

When exemplary embodiments of the system generate Network Adequacy reports which report one or more access parameters based on the health care service providers which are included in the Network, the system may generate a determination as to whether the addition of one or more available, non-participating providers improves conformance with CMS adequacy requirements. In one exemplary embodiment, at least one server retrieves health care service provider data from a database where the health care service provider data comprises data for health care service providers that are already a part of and those that are not yet a part of but may be added to a Medicare Advantage Network, analyzes whether the Network would be improved if at least one health care provider were added to the Network, if one health care provider that is currently in the Network were replaced with a health care provider that is not currently in the Network, if at least one health care provider currently in the network were dropped from the Network, etc. and reports how the Network would be improved in a Network adequacy Report that is generated by the server and sent to a remote computer for viewing. The report may identify the percentage of sample beneficiaries that meet a particular access parameter, before and after the proposed addition of a new health care provider.

In some exemplary embodiments, the system interactively reports the adequacy of a Medicare Advantage Network (and/or potential Medicare Advantage Network) to a system user accessing the system via a remote computer. In one such exemplary embodiment, at least one server receives a request from a remote computer to view information pertaining to a Medicare Advantage Network and in response to the request, retrieves health care service provider data from a database. In some exemplary embodiments, the server accesses a network adequacy reporting table which has been generated by the system and stored in a system database. In some instances, the health care service provider data retrieved from the database includes a list of health care service providers where some are already contracted and some are not contracted but available. The server may send the health care service provider data to the remote computer for viewing in such a way that indicates which health care service providers are currently contracted as well as send at least one Network Adequacy measure for viewing where the measure is based on the health care service providers which are currently contracted. The system user may be able to send instructions to at least one server of the system to recalculate at least one measure in response to a proposed change in the constitution of the health care service providers that are contracted. In one exemplary embodiment, the system user may select at least one non-included health care service provider by, for example, selecting it with a mouse or other electronic pointing device and may send an instruction to at least one server of the system to recalculate at least one measure based on the selected health care service provider's addition to the Medicare Network.

In another exemplary embodiment comprising interactive network adequacy reporting, the system may be instructed to recalculate at least one measure relating to network adequacy when a system user reconfigures a list of health care service providers sent by a system server for display at a remote computer. For example, a system server may send a display of health care service provider information to a remote computer for viewing. The display may comprise a first section where information for plan-included health care service providers is displayed and a second section for the display of non-included health care service provider information. The system user may be able to select the information of at least one plan-included health care service provider with a pointing device such as a mouse and “drag” it from the first display section to the second display section thereby causing an instruction to be sent to at least one server to recalculate at least one measure of network adequacy based on the service provider's removal from the Network. Similarly, a system user may be able to drag the information of a non-included health care service provider from the second display section to the first display section thereby causing an instruction to be sent to at least one server to recalculate at least one measure of network adequacy based on the addition of the health care service provider to the Network. Once the server receives an instruction to recalculate at least one measure from a remote computer, it may retrieve data from at least one database and recalculate the measure in accordance with the instruction then send the recalculated measure to be viewed by a remote computer.

When exemplary embodiments of the system send an interactive Network Adequacy report to a remote computer for viewing, the report may comprise a suggestion of an alteration to the Network which may improve at least one adequacy measure of the Network. For example, the server may analyze at least one adequacy measure for all the combinations of health care service providers which could potentially be utilized by a Medicare Advantage Network, compare the adequacy measures with at least one adequacy measure of the existing Medicare Advantage Network, and determine whether the adequacy measure of the existing Medicare Advantage Network could be improved by an alteration to the constitution of the plan's health care service providers. If at least one adequacy measure of the existing Medicare Advantage Network could be improved by altering the constitution of the plan's health care service providers, the server may cause the interactive Network Adequacy Report to display an indication that a better Network constitution has been detected. In preferred exemplary embodiments the server may also send an indication as to how the constitution of the Medicare Advantage Network should be amended in order to obtain the improved adequacy measure and may also display the improved adequacy measure that could be obtained.

In a preferred exemplary embodiment, at least one database storing health care service provider data may be accessed by one or more servers that are in communication with more than one remote computer. In such an embodiment, the system's communication with more than one remote computer may permit numerous system users to have access to the health care service provider and data stored in the database, review the data, and cause the generation of Medicare Network Adequacy reports. Some exemplary embodiments of the system comprise a user interface accessible at one or more remote computers where the user interface provides for the viewing of Network Adequacy reports. The user interface may be run by one or more servers of the system and accessed at one or more remote computers via the internet. In some exemplary embodiments a single interface may comprise an administrative console interface used to access an administrative console run by the system as well as a user interface where a system user may instruct the system to send a Network Adequacy Report to a remote computer for viewing. Log-in credentials may determine whether a system user has access to the administrative console and/or the user interface.

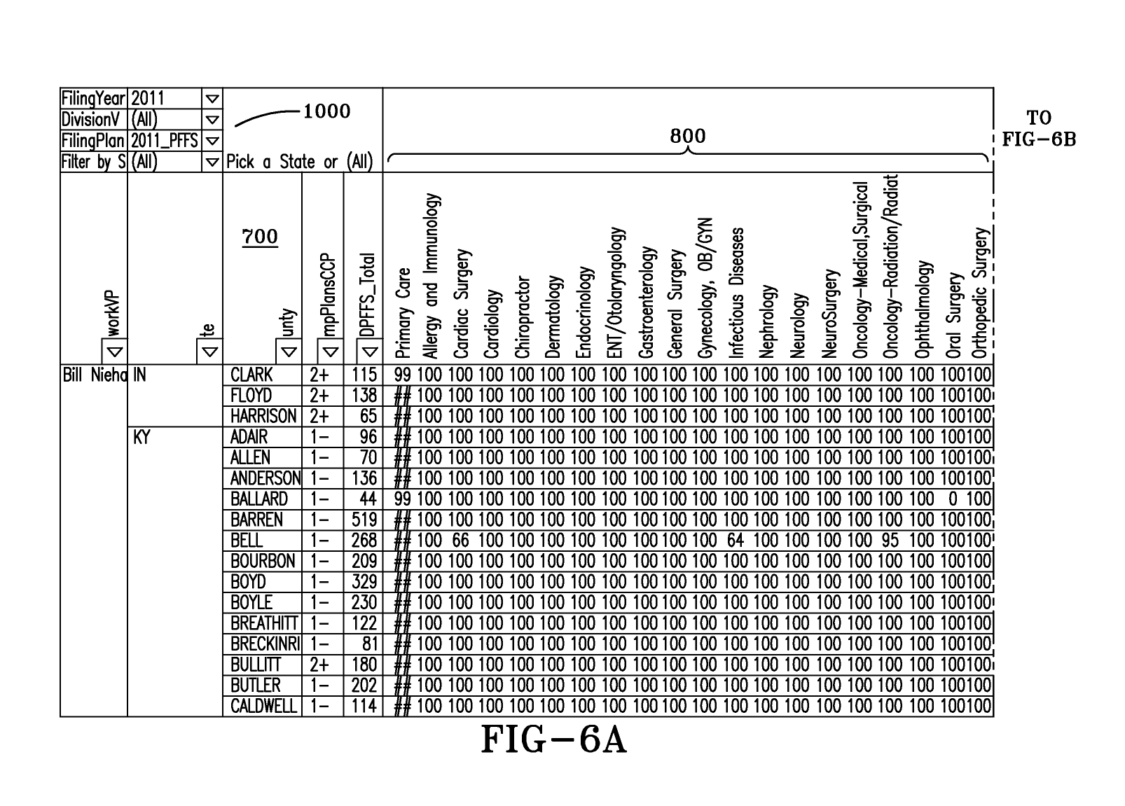

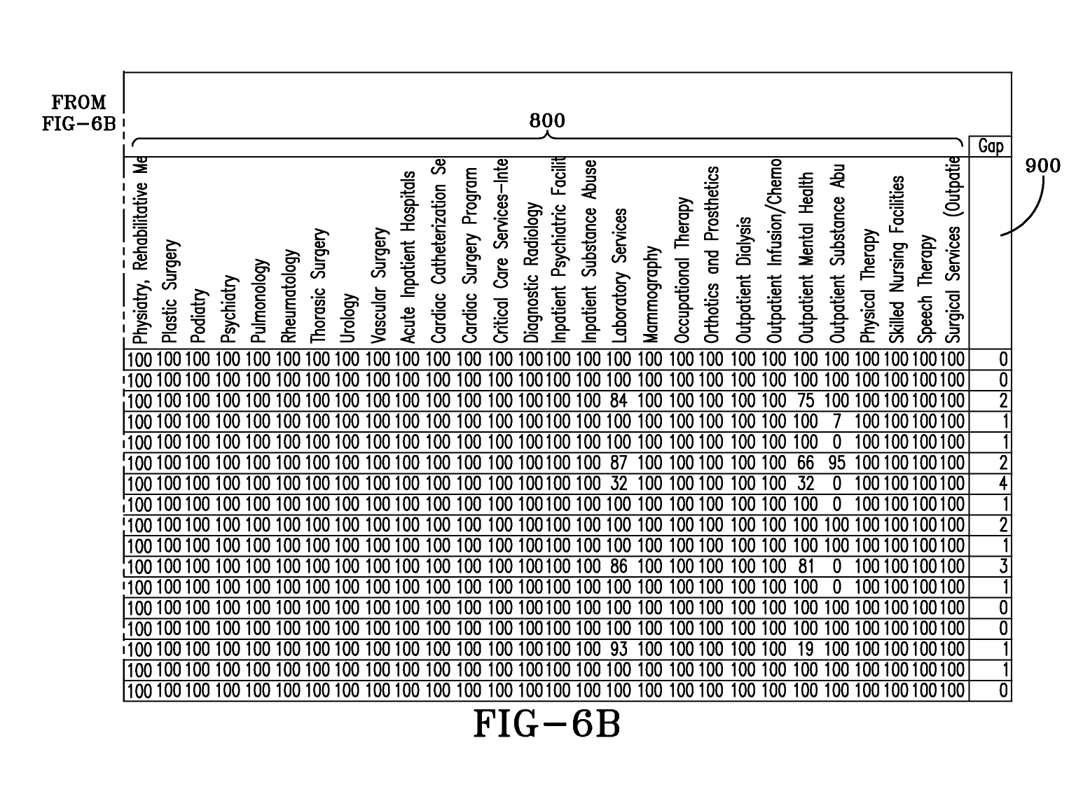

In some exemplary embodiments a system user may access a user interface to view at least one Network adequacy report that has been generated by the system. In an exemplary embodiment, once a user has used a remote computer to enter valid log-in credentials at a user interface of the system, a system server generates and sends a Network Adequacy Report to the remote computer for viewing. The Network Adequacy Report may be in the form of a worksheet which displays a summary of data for at least one Medicare Advantage Network and at least one measure of network adequacy for the network. In some exemplary embodiments, the Network Adequacy Report may comprise at least one County Summary page which displays data for the Medicare Advantage Networks of different counties. FIG. 6 shows an exemplary Network Adequacy Report County summary page. As can be seen, a county summary page may comprise a county column 700 which contains the name of counties with Medicare Advantage networks. The page may further contain at least one health care service column 800 for a type of health care service (cardiology, optometry, etc.) in which a measure of network adequacy is displayed for each of the listed counties. In the exemplary embodiment of FIG. 6, the displayed measure of network adequacy is the percentage of contracted health care service providers that meet a maximum distance requirement. As can be seen, a distinct adequacy measure is displayed for each type of health care service listed for each county. The report may also provide a gap column 900 which displays a gap value for each county. The gap value is the number of health care specialty divisions within the relevant county which fail to meet CMS requirements.

As shown in FIG. 6, a Network Adequacy Report may provide a system user with the ability to control the data that is displayed in the Report. For example, the Report may permit a user to enter at least one Medicare Network Parameter and send the Medicare Network Parameter to a system server which populates and/or repopulates the displayed Adequacy Report accordingly. In the exemplary embodiment shown in FIG. 6 a system user may send at least one Medicare Network Parameter to a system server for filtering the display of the Network Adequacy Report by selecting at least one Network Parameter from a drop-down menu 1000 displayed within the Report. In a preferred exemplary embodiment, a system user logs into the system via a user interface and the system sends a Network Adequacy Report to the user interface for viewing by the system user. The Network adequacy report sent by the system upon log-in may contain network adequacy data for every Medicare Advantage Network in which the system owner has an interest (for example, if the system owner is a private company that offers Medicare Advantage Plans the report may contain data pertaining to each Advantage network offered by the company). The system user viewing the Adequacy Report may be able to reduce the information displayed by the Network Adequacy Report by sending at least one Network Parameter to a system server. For example, a system user might select “State X” from a drop-down menu displayed in the Report or by the user interface. By selecting State X from the drop down menu, a system server may be instructed to repopulate the displayed Adequacy Report and limit the data within the Report to that pertaining to the Medicare Advantage Networks within State X. The Network Adequacy Report sent to the user interface for viewing by system users may be in the form of a worksheet such as an Excel worksheet.

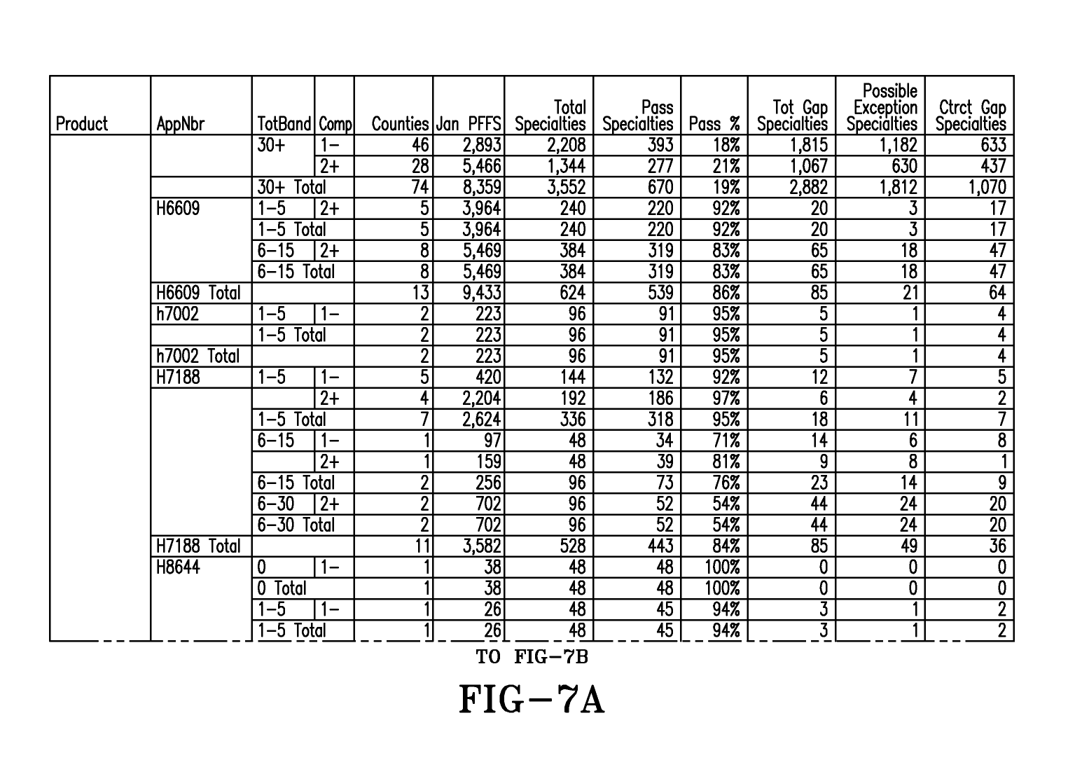

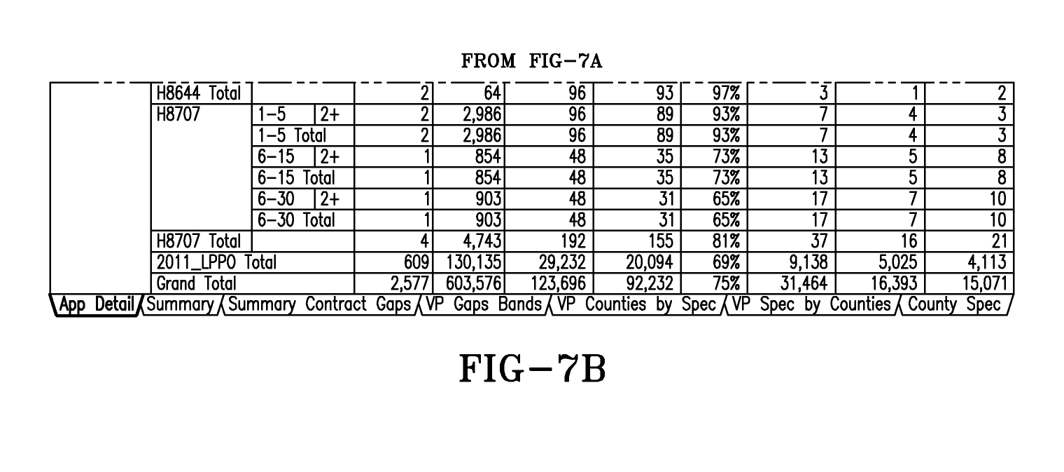

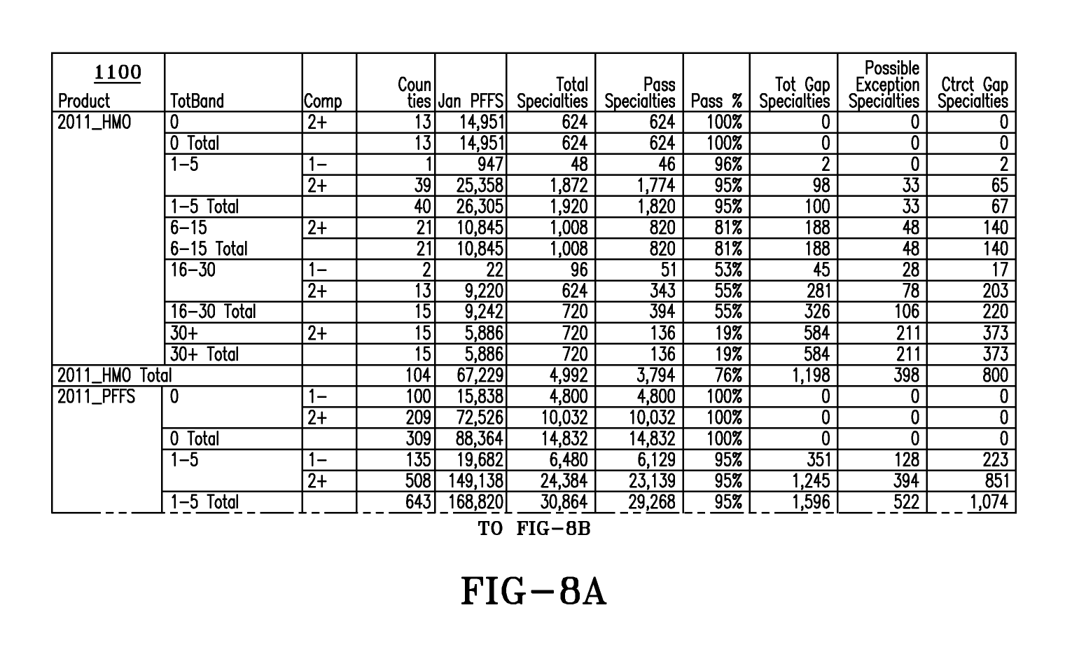

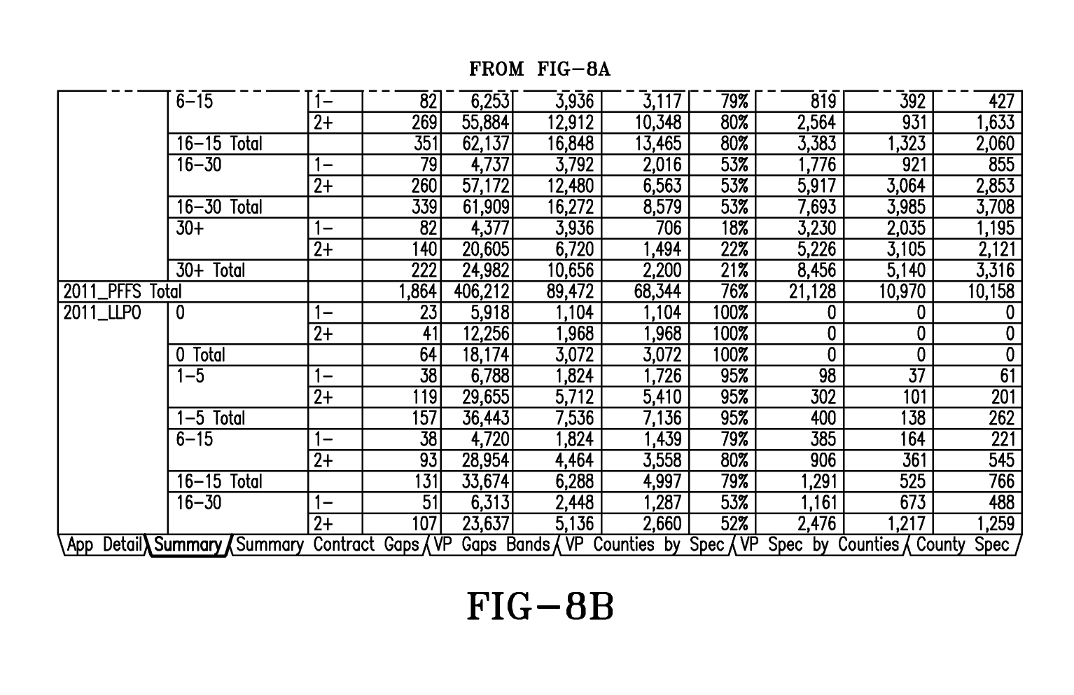

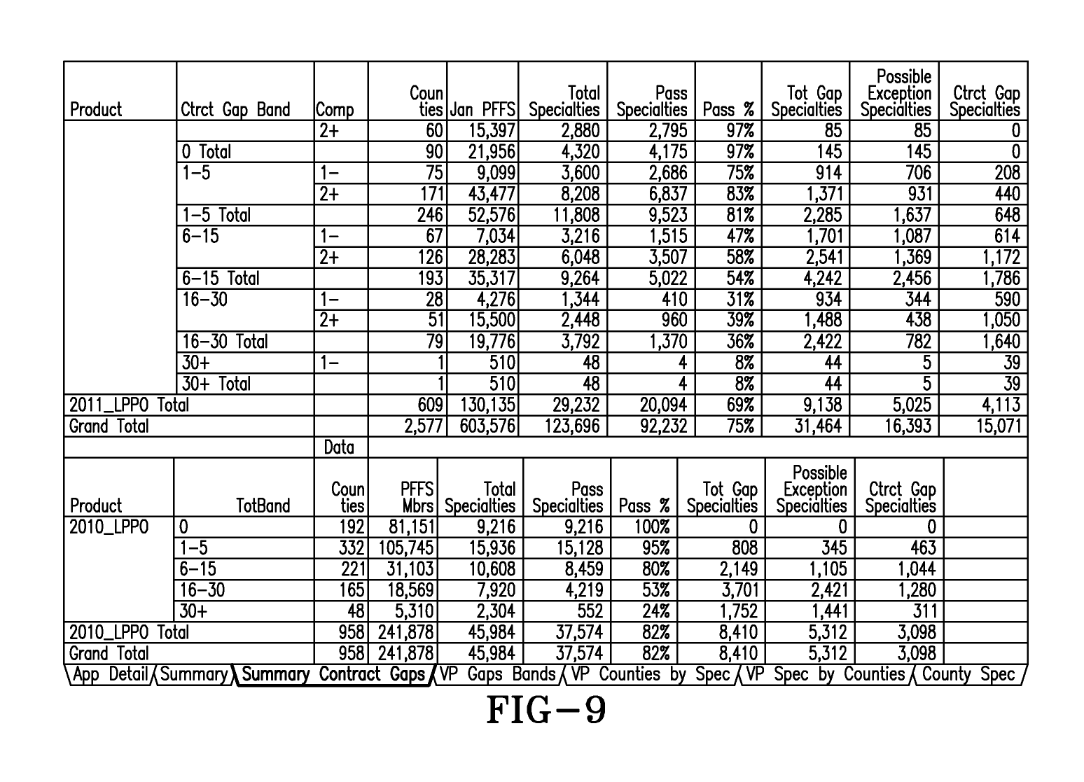

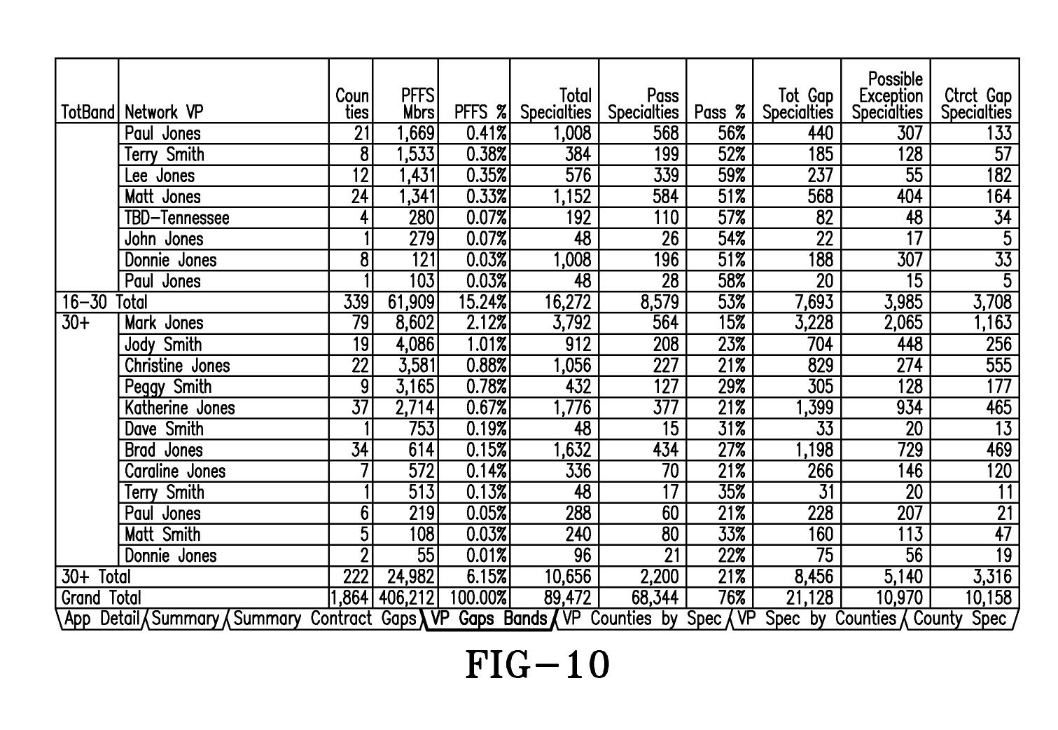

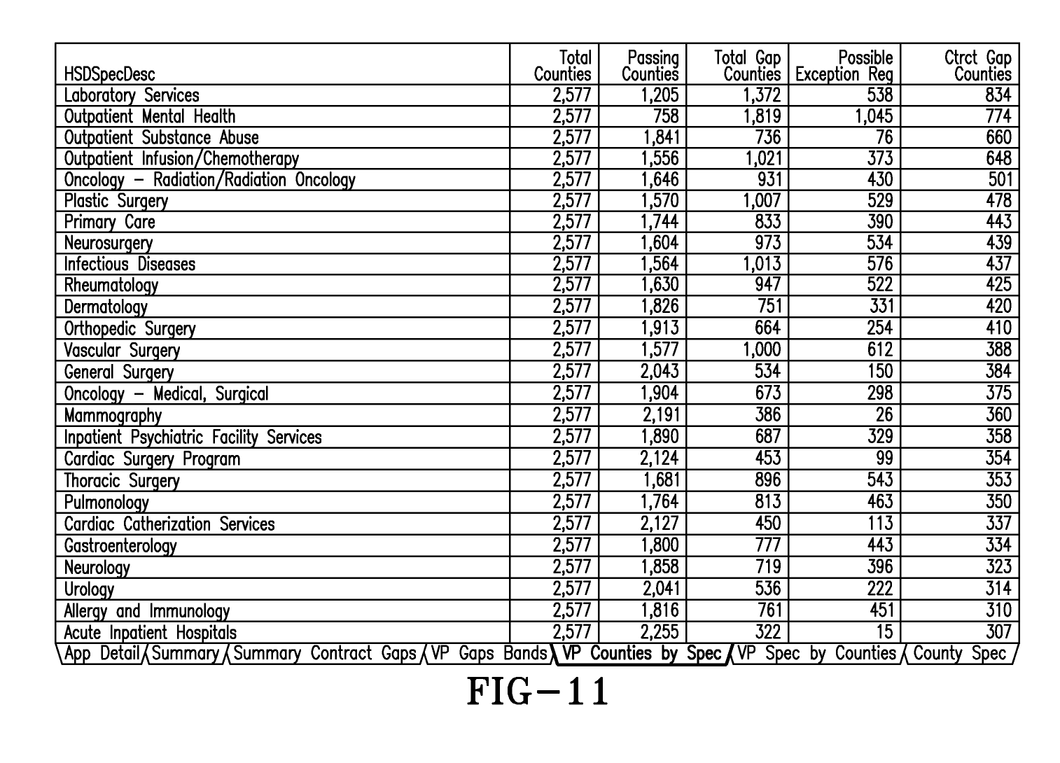

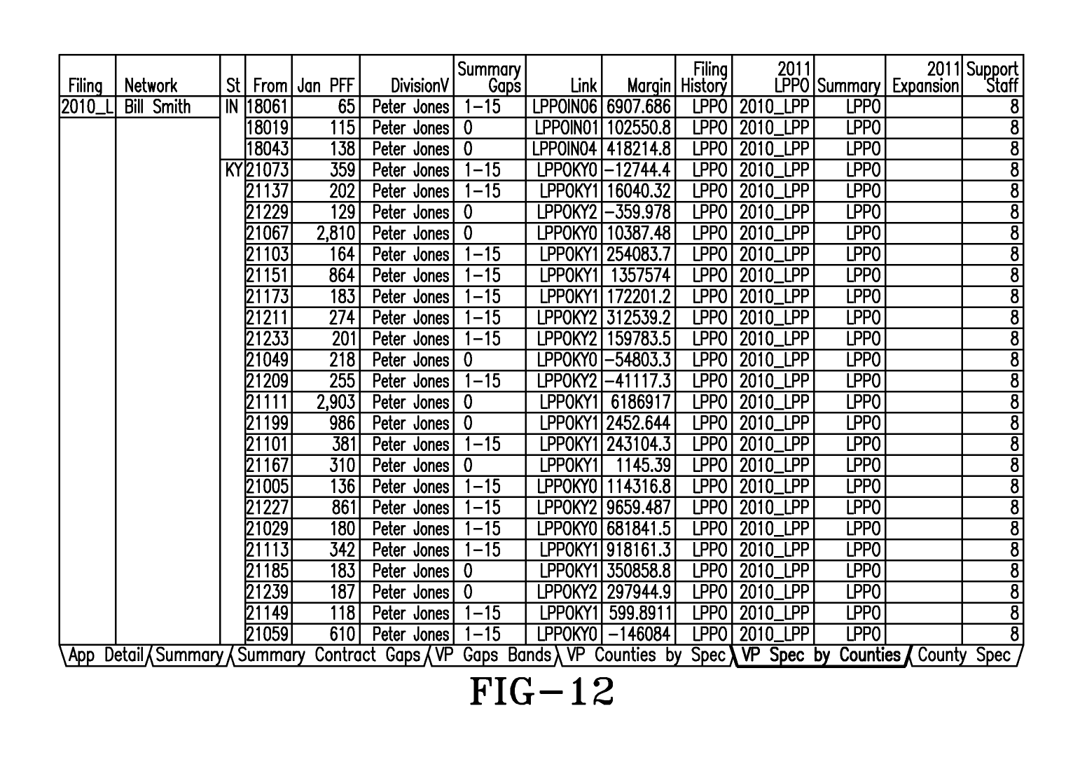

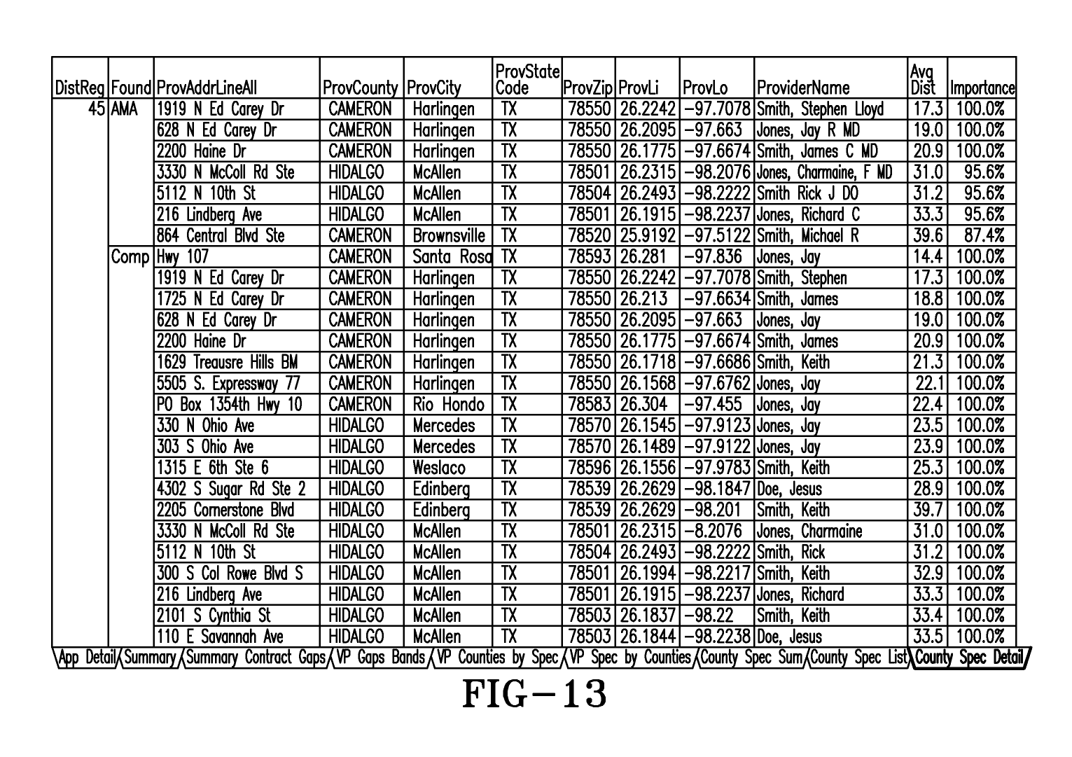

In a preferred exemplary embodiment, a system server populates an excel spreadsheet with Medicare advantage Network Plan data in order to create a Network Adequacy Report for viewing by a system user. The Excel Spreadsheet may comprise more than one sheet where each sheet may report different Medicare Advantage Network Plan data and/or where each sheet may organize Medicare Advantage Network Plan data in a different way. For example, a Network Adequacy Report may comprise an App Detail sheet, a Summary sheet, a Summary Contract Gap Sheet, a VP Gap Bands sheet, a VP Counties by Spec. sheet, a VP Spec. by Counties sheet, a County Spec. summary sheet, and a county spec detail sheet. FIG. 7 shows an exemplary embodiment of an App. Detail Sheet that may be generated by the system as part of a Network Adequacy Report. FIG. 8 shows an exemplary embodiment of a summary sheet that may be generated by the system as part of a Network Adequacy Report. In the exemplary summary page of FIG. 8, information is reported by product type and thus the page comprises a product column 1100 in which different type of Medicare Products (HMO, PFFS, PPO, etc.) are listed. FIG. 9 shows an exemplary embodiment of a summary contract gap sheet that may be generated by the system as part of a Network Adequacy Report. FIG. 10 shows an exemplary embodiment of a VP Gap bands sheet that may be generated by the system as part of a Network Adequacy Report. FIG. 11 shows an exemplary embodiment of a VP counties by spec. sheet that may be generated by the system as part of a Network Adequacy Report. FIG. 12 shows an exemplary embodiment of a VP spec by counties sheet that may be generated by the system as part of a Network Adequacy Report. FIG. 13 shows an exemplary embodiment of a county spec. detail sheet that may be generated by the system as part of a Network Adequacy Report.

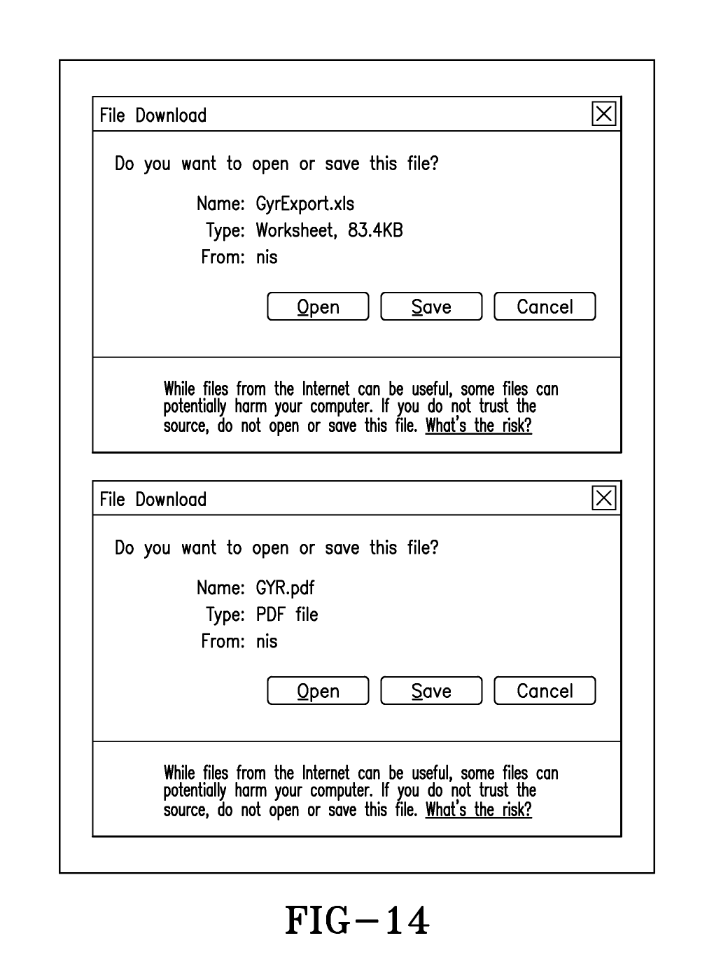

In some exemplary embodiments, a system user may be able to save a network adequacy report to a database at a remote computer and/or to a system database. For example, a user interface may display an icon along with a network generated adequacy report where the icon can be selected by the computer user in order to cause the system to generate a version of the adequacy report to be stored. In a preferred exemplary embodiment, a system user may save a network adequacy report in either PDF or Excel file format. FIG. 14 shows an exemplary embodiment of windows that may be generated by a system server once a system user has sent an instruction to the system to generate a version of the file for saving (by, for example, selecting an icon displayed by a user interface).

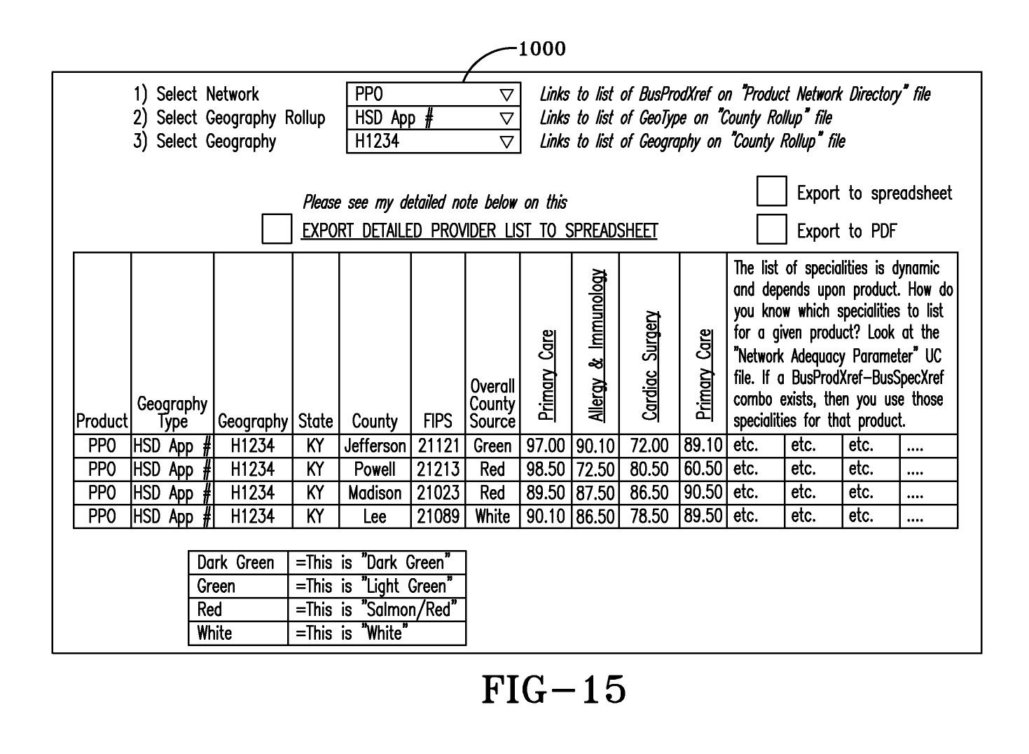

In some exemplary embodiments, the system generates a dynamic Medicare Network Adequacy Report. FIG. 15 shows one exemplary embodiment of a dynamic Medicare Network Adequacy Report that may be generated by the system. As shown in FIG. 15 a system user may be able to access the dynamic adequacy report and choose from at least one drop down menu 1000 to cause a Medicare Advantage Network Parameter to be sent to at least one server of the system. Once the drop down menu has been utilized to send at least one Medicare Advantage Network Parameter to a system server, the server may populate the displayed network adequacy report with network adequacy data that satisfies at least one parameter. For example, the exemplary embodiment of FIG. 15 gives system users the option of selecting a parameter from a network drop down menu, a geography rollup drop down menu, and a geography drop down menu.

In a preferred exemplary embodiment, such as that shown in FIG. 15 the contents of a dynamic Medicare network adequacy report vary depending on a product selection that has been made by a system user. For example, a system user may select “PPO” as a product from a “Select Network” drop-down menu displayed by a dynamic Medicare network adequacy report. Based on the product selected, the report displays adequacy measures for a predefined list of health care services or specialties. In the exemplary embodiment shown, the product that has been selected by a system user is “PPO” and the corresponding specialties displayed by the dynamic report for that product are Primary Care, Allergy and Immunology, Cardiac Surgery, and Cardiology. In some exemplary embodiments, when a system user has specified a product selection to the system, at least one server accesses a file that contains product data and corresponding specialty data for each product, locates the selected product within the file and extracts the corresponding specialty data for the product and sends it to the dynamic network adequacy report for viewing.

In some exemplary embodiments, the contents of a dynamic network adequacy report vary with multiple network parameters that have been specified to the system by a system user at a remote computer. In the exemplary embodiment of FIG. 15, the report contents may vary depending on product selected, geography rollup selection, and the geography selection sent to the system via the drop down menus displayed by the report. In a preferred exemplary embodiment, a dynamic network adequacy report displays at least one measure of network adequacy for a Medicare Advantage network. In the exemplary embodiment of FIG. 15, a report provides at least one measure of network adequacy for at least one Medicare Advantage Network that satisfies network parameters that have been sent to the system by a system user. In the exemplary embodiment of FIG. 15, network adequacy data is reported for four Medicare Advantage Networks (the networks existing in Jefferson, Powell, Madison, and Lee counties).

In some exemplary embodiments, a network adequacy report may utilize color or other graphical indicators to report network adequacy information. For example, Dark Green may be utilized to show that a Medicare Advantage Network measure exceeds CMS requirements, Light Green may be utilized to show that a Medicare Advantage measure meets CMS requirements, and Red (salmon) may be utilized to show that a Medicare Advantage measure does not satisfy CMS requirements. Colors may also be utilized to indicate whether reported measures exceed, meet, or fail requirements that are not established by CMS (for example, requirements or goals for a network that have been established by the system operator). FIG. 15 shows an exemplary embodiment of a Network Adequacy Report utilizing color to report network adequacy information.

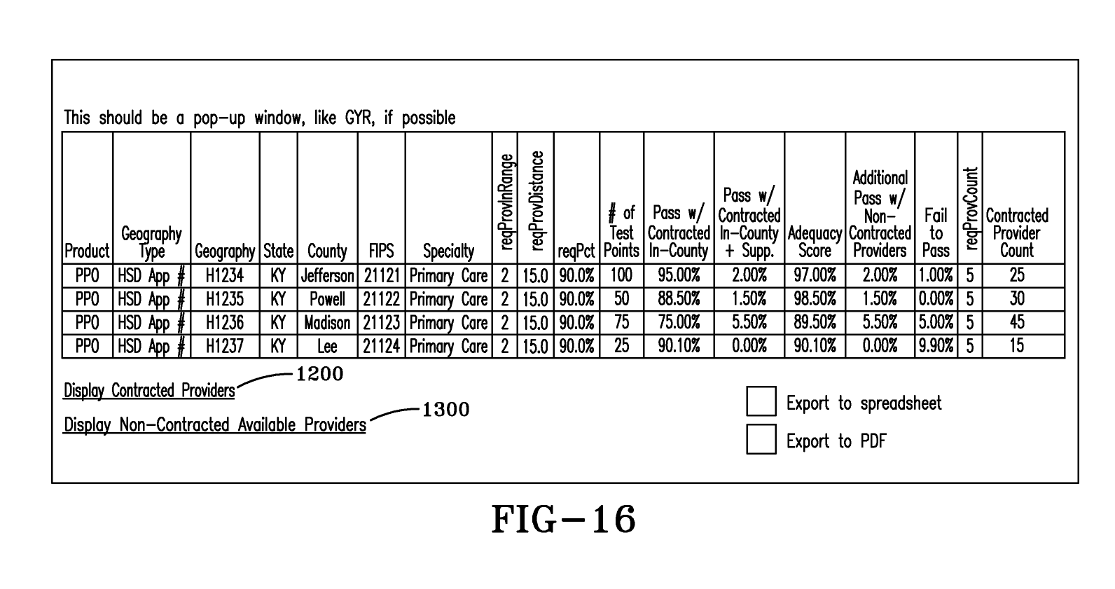

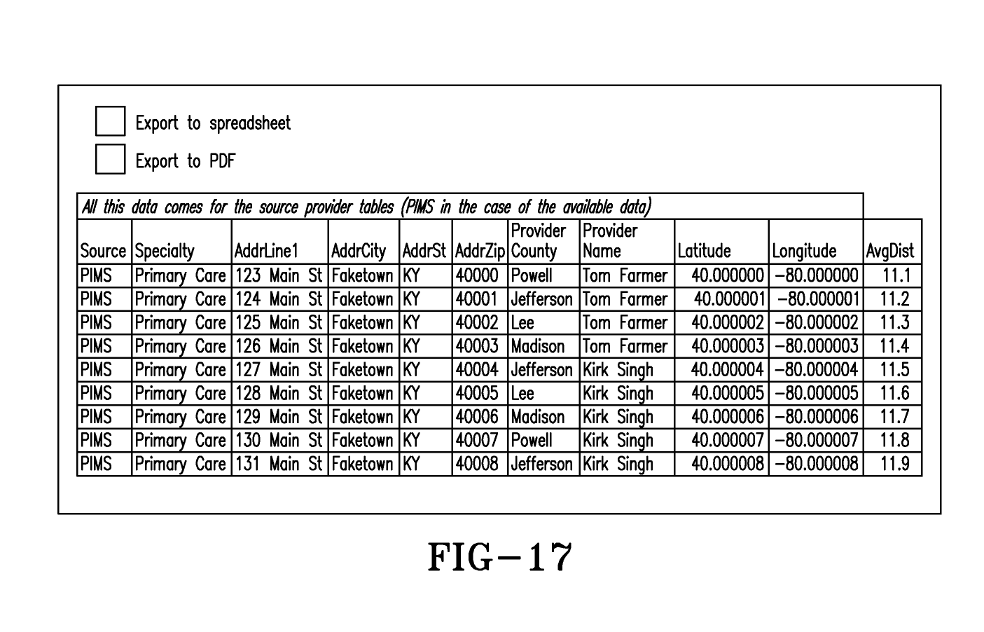

The system may permit a system user to view a network adequacy report and send an instruction to the system to generate a display of the health care service providers that are contracted and constitute at least one of the Medicare advantage networks represented within the report. FIG. 16 shows an exemplary embodiment of a Medicare Advantage Report which permits a system user to send an instruction to the system to generate and display of a list of contracted providers. In the FIG. 16 embodiment, the system user may send the instruction to the system by selecting the “Display Contracted Providers” option 1200 within the chart with a mouse or other electronic pointing device. When a system user has utilized an electronic pointer to send an instruction to the system, the instruction may be received by a system server which retrieves health care service provider data from at least one database in response to the instruction and sends the data to a remote computer for viewing. FIG. 17 shows an exemplary embodiment of a list of contracted health care service providers which may be generated by the system upon receiving an instruction from a system user. As shown, the system generated list may include information such as the health care service provider's specialty, address, county, name, geographical coordinates, and average distance information where the average distance information indicates the average distance of the service provider from network plan members (or as is discussed in more detail below, from test points that are intended to simulate the location of plan members within a Medicare advantage network).

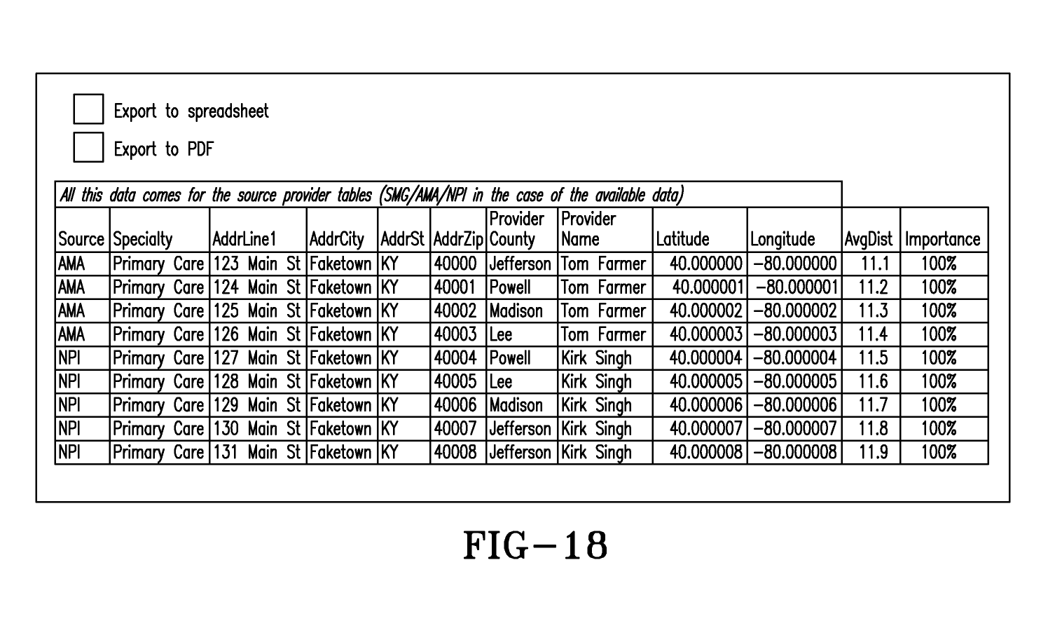

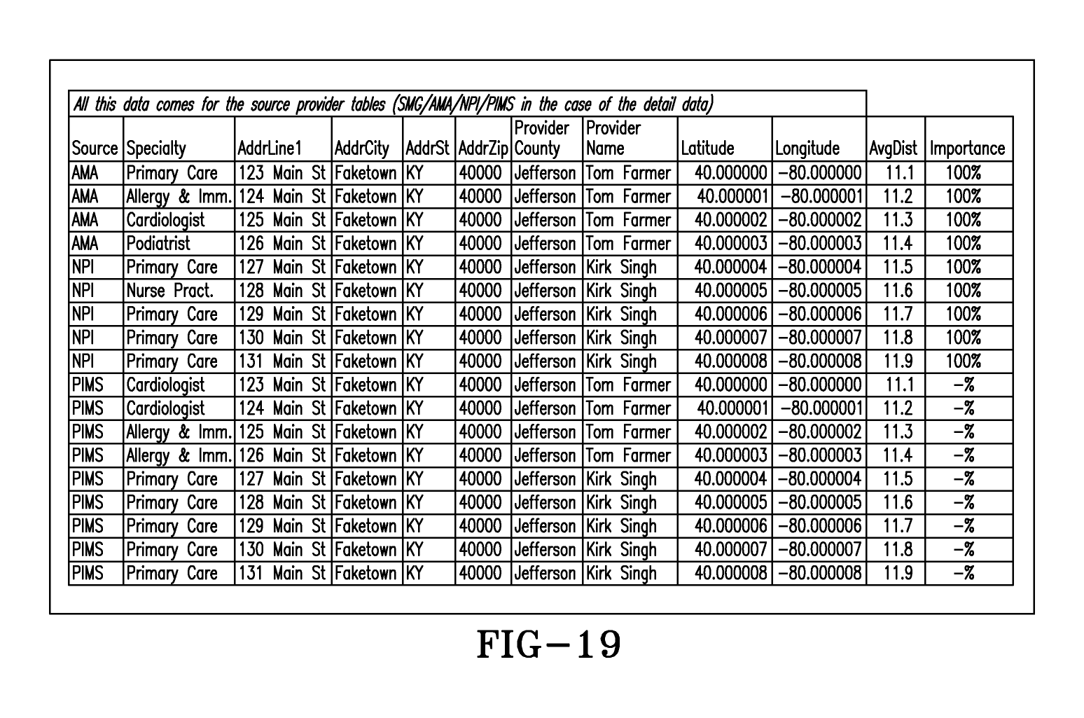

The exemplary embodiment of FIG. 16 further shows how a network adequacy report may permit a system user to send an instruction to the system to generate a display of non-participating health care service providers. The list preferably contains information for health care service providers that if contracted would improve a Medicare advantage network for which data is displayed in the Network Adequacy Report. For example, a listed non-participating health care service provider could permit a Network to become compliant by CMS standards. FIG. 18 shows an exemplary embodiment of a list of non-participating health care service providers which may be generated by the system upon receipt of an instruction sent by a system user. As shown, the list may include information such as the health care service provider's specialty, address, county, name, geographical coordinates, and average distance information where the average distance information indicates the average distance of the service provider from network plan members. As shown in FIG. 19, a network adequacy report generated by the system may contain a detail page which includes the information for contracted and non-contracted or non-participating health care service providers and display an indication of importance or ranking for contracting purposes. In the exemplary embodiment of FIG. 19, an available, non-participating health care service provider that could be contracted for a network to meet CMS requirements is listed with a 100% importance indicator.

When the system generates a list of contracted and/or non-participating health care service providers, it may obtain the data from source provider tables which have been generated by a system server and stored in at least one database of the system. In a preferred exemplary embodiment, the system is updated daily with health care service provider data. Once the system obtains the latest health care service provider data, a system server may assemble it into provider tables and store the provider tables in a database. A provider table may contain the data for health care service providers that offer their services to a certain geographical region (such as a county). The provider tables may be categorized and searched by a server of the network based on network parameter data which has been entered into the system by a system user (e.g., product, geographical parameters, etc.).

In some exemplary embodiments, the system utilizes test points to assemble a report of network adequacy. A test point may comprise a geographical coordinate utilized to simulate a plan member's location so that at least one measure of Medicare network adequacy may be determined. In one exemplary embodiment, the number of test points utilized by the system to test network adequacy varies with the classification of the county in which the Medicare Advantage Network in question is located. As has been discussed, CMS classifies counties and based on that classification, institutes various requirements for the Medicare Advantage Networks that are permitted to exist in the counties. In one exemplary embodiment, the system may utilize 100, 75, 50, or 25 test points to determine a measure of network adequacy for a Medicare Advantage Network where the number of test points utilized depends on CMS classification of the county at issue. FIG. 16 shows an exemplary embodiment of a Medicare Advantage Network Adequacy Report generated using test points.

In preferred exemplary embodiments, at least one system server retrieves health care service provider data from a database and applies at least one formula to assemble a Network Adequacy Reporting Table or a Network Adequacy Report. In some exemplary embodiments, the system utilizes a formula to determine the distance between two points. For example, the system may utilize a formula to determine the distance between the location of a health care service provider and the location of a Medicare network plan member in order to determine if CMS standards are satisfied. The system may also utilize a formula to determine the distance between a health care service provider and a test point which represents a theoretical Medicare Advantage Plan Member. The system may determine the distance between two points (latitude, longitude), Point 1 (B2, C2) and Point 2 (D2, E2) via the following formula:

$$Distance = 3963.1 \times ACOS ( SIN ( \frac{B 2}{52.29577951} ) \times SIN ( \frac{D 2}{52.29577951} ) + COS ( \frac{B 2}{57.29577951} ) \times COS ( \frac{D 2}{57.29577951} ) \times COS ( \frac{E 2}{57.29577951} - \frac{C 2}{57.29577951} ) ) . \qquad Equation 3$$

Further, the system may adjust the Formula for an approximation of drive distance by multiplying the result of Equation 3 as follows:

$$( Equation 3 ) \times ( 1.05 + 0.35 \times ( 1 - Tan ( abs ( 0.785398 - atan ( abs ( \frac{( B 2 - D 2 )}{( C 2 - E 2 )} ) ) ) ) ) ) . \qquad Equation 4$$

In some exemplary embodiments, it may be necessary to adjust Equations 3 and 4 for out-of range values such as division by 0 or ArcCos of a greater than 1 value, which may cause out of range results. Formulas 3 and 4 preferably assume that the latitude and longitude values are floating point values in degrees e.g., (38.25,−85.75).

In preferred exemplary embodiments, the system uses equations 3 and 4 to determine at least one measure of network adequacy for a Medicare Advantage Network. A measure of network adequacy may be determined by the system for a variety of health service specialties (i.e., primary care, cardiology, etc.) that are included in a county's Medicare Advantage Network.

In some exemplary embodiments, at least one system server retrieves health care service provider data from a database, where the data comprises the geographical location (latitude, longitude) of at least one health care service provider, and uses Equations 3 and 4 in conjunction with at least one Test Point to determine at least one measure of network adequacy and populates a network adequacy reporting table with at least one measure. Utilizing the example adequacy determination for county “01234” provided above, the system may assemble a network adequacy reporting table detailing the results. For example, the system may assemble a table comprising data indicating that the county currently does not pass CMS adequacy requirements, but could pass CMS adequacy requirements if two health care service providers (A1 and A2) are contracted to be a part of the Medicare Advantage Network. The report may list the health care service providers (A1 and A2) which are available and need to be contracted in order for the Network to be considered adequate by CMS. However, as shown by FIG. 16, in some exemplary embodiments the report may not contain a list of available health care service providers but might permit a system user to instruct the system to generate such a list. There are a variety of ways a Network Adequacy Report generated by the system may display information.

In some exemplary embodiments, a system for generating a network adequacy report may be utilized in conjunction with or as part of a system for preparing a health service delivery (“HSD”) table. An exemplary system for preparing and filing HSD tables may comprise one or more servers running a Network Adequacy Tool that analyzes data within a final file to determine whether the network represented within the file is adequate. The tool is preferably an automated tool that analyzes data within Final File tables of the system to determine whether or not minimum Medicare requirements are being met.

In one exemplary embodiment, the network adequacy tool analyzes the data contained within a Final File of the system and determines whether the requirements of CMS would be met by the network of providers included in the table. The network adequacy tool may analyze the provider data within the final file tool to determine whether the network access parameters and minimum provider counts established by CMS have been satisfied. In cases where the analysis shows the network access parameters and minimum provider counts established by CMS have not been satisfied, the network adequacy tool may search through health care service provider data within a system database to find providers that satisfy the network's “supplemental provider” criteria (by utilizing county coverage data for example) to tag providers for the network in question until the access parameters and minimum provider counts have been satisfied. When a system for generating a network adequacy report is utilized in conjunction with a system and method for preparing and filing HSDS tables, the system for generating a network adequacy report may utilize calculations of network adequacy made by the network adequacy tool to populate a table with at least one measure of network adequacy which may be sent to a remote computer for viewing as a network adequacy report which assists a system user in preparing an HSDS table for filing with CMS.

Having shown and described exemplary embodiments of the present invention, those skilled in the art will realize that many variations and modifications may be made to the described invention and still be within the scope of the claimed invention. Thus many of the elements indicated above may be altered or replaced by different elements which will provide the same or substantially the same result and fall within the spirit of the claimed invention. It is the intention, therefore, to limit the invention only as indicated by the scope of the following claims:

### APPENDIX

The following is an example of how the system might determine a measure of network adequacy using equations 3 and 4 for sample Network “MedPPO” in county “01234” for HSD specialty “Primary Care.” The following requirements may apply in determining a measure of network adequacy:

| | |
|---|---|
| Maximum Distance | 30 Miles |
| For Each Test Point, Health Care Service | 2 |
| Providers Needed | |
| Percentage of Test Points Needed to Pass CMS | 90% |
| Requirements | |
| Total County Health Care Service Providers | 4 (this may be |
| Needed | determined utilizing |
| | Equations 1 and 2) |

The system may perform a gap calculation (a calculation as to whether the county passes CMS requirements) using four case test points as follows:

### Case Test Point 1

For Test Point T1 with sample count 4 at (40.07,−87.00)

Contracted Providers in County=Provider P1 (40.07,−86.05), Provider P2 (40.17,−86.65), Provider P3 (40.27,−86.95)

STEP 1: the system analyzes the distance of the in-county health care service providers from test point 1 using Equations 3 & 4:
a) Distance Between T1 and P1=53 miles (based on Quest Drive Distance Formula)>Fail
b) Distance Between T1 and P2=24 miles (Less than Maximum Distance=30 miles)>Pass (Maximum Distance)
c) Distance Between T1 and P3=17 miles>Pass (Maximum Distance)

STEP 2: the system determines whether the Medicare Advantage Network meets CMS standards for Test Point 1 by comparing the number of health care service providers within the Maximum distance of 30 miles with the number of health care service providers required to be within the maximum distance per test point (in this example 2 health care service providers are required per test point).

Because 2 health care service providers (P2 and P3) are within 30 miles of Test Point 1, Test Point 1 passes adequacy requirements with in-county health care service Providers (health care service providers physically located within County “01234”)

### Case Test Point 2

For Test Point T2 with sample count 3 at (40.07, −87.00)

Contracted Health Care Service Providers in County=Provider P3 (40.07,−86.05)

Contracted Health Care Service Providers outside of County=Provider P4 (40.17,−86.65) and Provider P5 (40.27,−86.95)

STEP 1: the system analyzes in-county health care service providers using Equations 3 & 4:
a) Distance Between T2 and P3=53 miles>Fail

STEP 2: the system analyzes out-of-county health care service providers using Equations 3 & 4:
a) Distance Between T2 and P4=24 miles (Less than Maximum Distance=30 miles)>Pass
b) Distance Between T2 and P5=17 miles>Pass

STEP 3: the system determines whether the Medicare Advantage Network meets CMS standards for Test Point 2 by comparing the number of health care service providers within the Maximum distance of 30 miles with the number of health care service providers required to be within the maximum distance per test point (in this example, 2). Because two health care service providers are within 30 miles of Test Point 2, the test point passes.

### Case Test Point 3

For Test Point T3 with sample count 2 at (40.07 −87.00)

Contracted Health Care Service Providers in County=Provider P6 (40.07,−86.05)

Contracted Health Care Service Providers outside County=Provider P7 (40.17,−86.15), Provider P8 (40.27,−86.25)

Available Health Care Service Providers in or outside County=Provider A1 (40.17,−86.65), Provider A2 (40.27,−86.95)

STEP 1: the system determines the distance of in-county health care service providers from Test Point 3 using Equations 3 and 4:
a) Distance Between T3 and P6=53 miles>Fail

STEP 2: the system determines the distance of out-of-county health care service providers from Test Point 3 using Equations 3 and 4:
a) Distance Between T3 and P7=51 miles>Fail
b) Distance Between T3 and P8=50 miles>Fail

STEP 3: the system considers health care service providers that are available but have not yet been contracted to be a part of the Medicare Advantage Network and determines the distance of the available providers from Test Point 3:
a) Distance Between T3 and A1=24 miles>Pass
b) Distance Between T3 and A2=17 miles>Pass

STEP 4: the system determines whether the Medicare Advantage Network meets CMS standards for Test Point 3 by comparing the number of health care service providers within the Maximum distance of 30 miles with the number of health care service providers required to be within the maximum distance per test point (in this example, 2). In this case, Test Point 3 will pass if A1 and A2 are contracted to join the Network because they are both within the 30 mile maximum distance.

### Case Test Point 4

For Test Point T4 with sample count 1 at (40.07, −87.00)

Contracted Health Care Service Providers in County=Provider P6 (40.07,−86.05)

Contracted Health Care Service Providers outside of County=Provider P7 (40.17,−86.15), Provider P8 (40.27,−86.25)

Available Health Care Service Providers in or outside County=Provider A1 (40.17,−86.15), Provider A2 (40.27,−86.25)

STEP 1: the system determines the distance of in-county health care service providers from Test Point 4 using Equations 3 and 4:
a) Distance Between T4 and P6=53 miles>Fail

STEP 2: the system determines the distance of out-of-county health care service providers from Test Point 3 using Equations 3 and 4:
a) Distance Between T4 and P7=51 miles>Fail
b) Distance Between T4 and P8=50 miles>Fail

STEP 3: the system considers health care service providers that are available but have not yet been contracted to be a part of the Medicare Advantage Network and determines the distance of the available providers from Test Point 3:
a) Distance Between T4 and A1=51 miles>Fail
b) Distance Between T4 and A2=50 miles>Fail

STEP 4: the system determines whether the Medicare Advantage Network meets CMS standards for Test Point 4 by comparing the number of health care service providers within the Maximum distance of 30 miles with the number of health care service providers required to be within the maximum distance per test point (in this example, 2). In this case, Test Point 4 fails because there are no health care service providers within the 30 mile maximum distance.

The foregoing calculations produce the following results for this example:

Test Point T1(4 Sample Points) Pass With In-County providers P2, P3

Test Point T2(3 Sample Points) Pass With Supplemental providers P4, P5

Test Point T3(2 Sample Points) Pass With Available providers A1, A2

Test Point T4(1 Sample Point) Fail

Total Sample Points in County “01234”=4+3+2+1=10

Total Sample Points that Pass based on In-County health care service providers=4 (from Test Point 1)=40% of total sample points

Total Sample Points that Pass based on In-County and Supplemental health care service providers=4+3 (from Test Points 1 and 2)=7=70% of total sample points=Fail based on the 90% requirement

Total Sample Points that Pass based on In-County and Supplemental+Available health care service providers=4+3+2=9=90% of total sample points

The total Contracted Providers available for Test Points in the County=P2, P3, P4, P5=4 Providers which means the Network satisfies the requirement that there be 4 contracted health care service providers in the Network.

The Net Result in this exemplary calculation is that the County Fails to satisfy adequacy requirements, but could pass if Available Target Providers (A1 and A2) are contracted.

1. A computerized method for analyzing a health plan's medical network with a geographic region comprising:
(a) storing in at least one computerized database health care service provider data comprising for each of a plurality of health care service providers:
(i) location data for said health care service provider for use in calculating an adequacy measure;
(ii) specialty type data for said health care service provider for use in calculating an adequacy measure; and
(iii) an availability indicator for said health care service provider that indicates whether said health care service provider is contracted or is not contracted in said health plan's medical network;
(b) receiving at a server member population data for a member population to be served in said health plan's medical network of said health care service providers;
(c) receiving at said server access parameters for measuring compliance of said health plan's medical network to said access parameters for said member population;
(d) calculating at said server an adequacy measure for said health plan's medical network in relation to:
(i) said access parameters; and
(ii) a test point simulating a geographic location of member beneficiaries within said geographic region;
(e) in response to determining said adequacy measure fails to meet said access parameters
(i) locating in said at least one computerized database according to said availability indicator a plurality of non-contracted health care service providers within said geographic region that are not contracted in said health plan's medical network;
(ii) receiving at said server a selection of one of said plurality of non-contracted health care service providers;
(f) adding said at least one non-contracted health care service provider to said health plan's medical network;
(g) quantifying at said server an impact on said adequacy measure for said health plan's medical network in relation to
(i) said access parameters and
(ii) said test point simulating said geographic location of said member beneficiaries within said geographic region
if said non-contracted health care service provider is added to said health plan's medical network; and
(h) generating at said server for display to a computer user a report comprising said impact on said adequacy measure.

2. The method of claim 1 wherein calculating at said server an adequacy measure comprises applying a formula for calculating a required minimum number of health care service providers.

3. The method of claim 1 wherein calculating at said server an adequacy measure comprises applying a formula for calculating a minimum provider ratio.

4. The method of claim 1 wherein calculating at said server an adequacy measure comprises applying a formula for calculating a distance between said test point and a health care service provider.

5. The method of claim 1 wherein calculating at said server an adequacy measure comprises applying a formula for calculating an approximate driving distance from said test point to a health care service provider.

6. The method of claim 1 wherein said adequacy measure is calculated for a specified health service specialty.

7. The method of claim 1 wherein said adequacy measure is calculated for a specified health service product.

8. A computerized method for analyzing a health plan's medical network within a geographic region comprising:
(a) storing in at least one computerized database health care service provider data comprising for each of a plurality of health care service providers:
(i) location data for said health care service provider for use in calculating an adequacy measure;
(ii) specialty type data for said health care service provider for use in calculating an adequacy measure; and
(iii) an availability indicator for said health care service provider that indicates whether said health care server provider is contracted or not contracted in said health plan's medical network;
(b) receiving at a server member population data for a member population to be served in said health plan's medical network of said health care service providers;
(c) receiving at said server access parameters for measuring compliance of said health plan's medical network to said access parameters for said member population;
(d) calculating at said server an adequacy measure for said health plan's medical network in relation to:
(i) said access parameters; and
(ii) a test point simulating a geographic location of member beneficiaries within said geographic region;
(e) generating at said server for display to a computer user a report comprising said adequacy measure for said health plan's medical network;
(f) locating in said at least one computerized database according to said availability indicator a plurality of non-contracted health care service providers that are not contracted in said medical network;
(g) generating at said server a list of non-contracted health care service providers within said geographic region;
(h) receiving at said server from said computer user a request to add one of said plurality of non-contracted health care service providers from said list to said health plan's medical network;
(i) quantifying at said server an impact on said adequacy measure for said health plan's medical network in relation to
(i) said access parameters and
(ii) said test point simulating said geographic location of said member beneficiaries within said geographic region
if said at least one non-contracted health care service provider is added to said health plan's medical network; and
(j) generating at said server for display to a computer user a report comprising said impact on said adequacy measure for said health plan's medical network following addition of said at least one non-contracted health care service provider to said health plan's medical network.

9. The method of claim 8 wherein calculating at said server an adequacy measure comprises applying a formula for calculating a required minimum number of health care service providers.

10. The method of claim 8 wherein calculating at said server an adequacy measure comprises applying a formula for calculating a minimum provider ratio.

11. The method of claim 8 wherein calculating at said server an adequacy measure comprises applying a formula for calculating a distance between said test point and a health care service provider.

12. The method of claim 8 wherein calculating at said server an adequacy measure comprises applying a formula for calculating an approximate driving distance from said test point to a health care service provider.

13. The method of claim 8 wherein said adequacy measure is calculated for a specified health service specialty.

14. The method of claim 8 wherein said adequacy measure is calculated for a specified health service product.

15. A computerized method for analyzing a health plan's medical network comprising:
(a) storing in a computerized database health care service provider data comprising for each of a plurality of health care service providers:
(i) location data for said health care service provider for use in calculating an adequacy measure;
(ii) specialty type data for said health care service provider for use in calculating an adequacy measure; and
(iii) an availability indicator for said health care service provider that indicates whether said health care server provider is contracted or is not contracted in said health plan's medical network;
(b) receiving at a server member population data for a member population to be served in said health plan's medical network of said health care service providers;
(c) receiving at said server access parameters for measuring compliance of said health plan's medical network to said access parameters for said member population;
(d) calculating at said server an adequacy measure for said health plan's medical network in relation to:
(i) said access parameters; and
(ii) a test point simulating a geographic location of member beneficiaries within said geographic region;
(e) in response to determining said adequacy measure fails to conform to said access parameters:
(i) locating in said at least one computerized database according to said availability indicator and said location data a plurality of non-contracted health care service providers that are not contracted in said health plan's medical network; and
(ii) generating a rank ordered list of said non-contracted health care service providers within said geographic region;
(f) receiving at said server from said computer user a request to add at least one non-contracted health care service provider from said list to said health plan's medical network;
(g) quantifying at said server a measure of improvement in said adequacy measure for said health plan's medical network in relation to
(i) said access parameters and
(ii) said test point simulating said geographic location of said member beneficiaries within said geographic region
when said at least one non-contracted health care service provider is added to said health plan's medical network; and
(h) generating at said server for display to a computer user a report comprising said measure of improvement in said adequacy measure for said health plan's medical network.

16. (canceled)
